(19) United States
(12) Patent Application Publication
(10) Pub. No.: US 2023/0419491 A1
(43) Pub. Date: Dec. 28, 2023

(54) ATTENTION-BASED MULTIPLE INSTANCE LEARNING FOR WHOLE SLIDE IMAGES

(71) Applicant: Genentech, Inc., South San Francisco, CA (US)

(72) Inventor: Fang-Yao HU, San Mateo, CA (US)

(21) Appl. No.: 18/463,585

(22) Filed: Sep. 8, 2023

Related U.S. Application Data
(63) Continuation of application No. PCT/US2022/020059, filed on Mar. 11, 2022.
(60) Provisional application No. 63/160,493, filed on Mar. 12, 2021.

(51) Int. Cl. G06T 7/00; G06T 7/11; G06V 20/69; G06V 20/70; G06V 10/82; G16H 30/40

(52) U.S. Cl. CPC G06T 7/0012; G06T 7/11; G06V 20/698; G06V 20/695; G06V 20/70; G06V 10/82; G16H 30/40; G06T 2207/20021; G06T 2207/30204; G06T 2207/20084; G06T 2207/30004; G06T 2207/10056; G06T 2207/30024

(57) ABSTRACT

In one embodiment, a method includes, receiving a whole slide image and segmenting the whole slide image into multiple image tiles. The method includes generating a feature vector corresponding to each tile of the plurality of tiles, wherein the feature vector for each of the tiles represents an embedding for the tile. The method includes computing a weighting value corresponding to each embedding feature vector using an attention network. The method includes computing an image embedding based on the embedding feature vectors, wherein each embedding feature vector is weighted based on the weighting value corresponding to the embedding feature vector. The method includes generating a classification for the whole slide image based on the image embedding.

### CROSS-REFERENCE TO RELATED APPLICATION(S)

This application claims the benefit of and the priority to U.S. Provisional Application No. 63/160,493 entitled “ATTENTION-BASED MULTIPLE INSTANCE LEARNING FOR WHOLE SLIDE IMAGES” and filed on Mar. 12, 2021, which is hereby incorporated by reference in its entirety for all purposes.

### TECHNICAL FIELD

This disclosure generally relates to tools for analyzing and classifying digital pathology images.

### BACKGROUND

Whole Slide Images (WSI) result from scans of images of samples or from digital-native scans. A scan, and the corresponding WSI, is often very large, for example 100,000 pixels by 100,000 pixels in each of several color channels, making it difficult to efficiently analyze WSI on a holistic level using traditional computational methods. Current approaches to handle the large formats of WSI include segmenting the WSI into smaller portions and performing parallel analysis using multiple processors or otherwise distributed processing. Segmenting and distributed processing may be useful to gather understanding of the discrete portions but cannot generate an understanding of the WSI as a whole.

A pathologist or other trained specialist will often evaluate a WSI for evidence of abnormalities in the depicted tissue. Labeling for WSI tends to refer to the entire image and not, for example, to a specific portion of an image. For example, a pathologist may identify a tissue abnormality (e.g., a tumor) in an image of a lung and label the image as “abnormal.” In most cases, however, the pathologist will not annotate the image to specify where in the image the tissue abnormality appears. This “all or nothing” labelling style is less useful for training computer-implemented algorithms to evaluate WSI. However, even under whole-image labelling, pathologist analysis is time consuming. To have pathologists re-evaluate old samples to mark individual locations is prohibitively time consuming. Moreover, many conditions are not mutually exclusive, so a single WSI may indicate multiple conditions simultaneously which may require multiple specialists to review the image simultaneously to ensure that all abnormal conditions are labeled.

Accordingly, a desire exists for WSI labels or annotations that provide refinement past a binary labeling of images as “normal image” or “abnormal image.” In addition, a desire exists for such annotations to pinpoint portions of an image or locations in an image that depict an abnormality.

### SUMMARY OF PARTICULAR EMBODIMENTS

In particular embodiments, a computer-implemented method includes, receiving or otherwise accessing a whole slide image and segmenting the whole slide image into multiple tiles. In particular, the whole slide image may be a large format image and the size of the segmented tiles may be selected to facilitate efficient management and processing. The method includes generating an embedding feature vector corresponding to each tile of the plurality of tiles. In particular embodiments, the embedding feature vectors are generated using a neural network trained using natural images. The method includes computing a weighting value corresponding to each embedding feature vector using an attention network. The method includes computing an image embedding from the embedding feature vectors. Each embedding feature vector is weighted from the weighting value corresponding to the embedding feature vector. In particular embodiments, the method further includes normalizing the weighting values prior to computing the image embedding. The method includes generating a classification for the whole slide image from image embedding. The classification for the whole slide image may indicate the presence of one or more biological abnormalities in tissue depicted in the whole slide image, include hypertrophy, Kupffer cell abnormalities, necrosis, inflammation, glycogen abnormalities, lipid abnormalities, peritonitis, anisokaryosis, cellular infiltration, karyomegaly, microgranuloma, hyperplasia, or vacuolation. The classification for the whole slide image may include an evaluation of a potentially toxic event associated with tissue depicted in the whole slide image. In particular embodiments, the computer may compute weighting values corresponding to each embedding feature vector using multiple attention networks and generate a respective classification for the whole slide image from each attention network. In particular embodiments, the classification indicates the whole slide image depicts one or more abnormalities associated with the tissue depicted in the whole slide image. In particular embodiments the method includes providing the classification for the whole slide image to a pathologist for verification.

In particular embodiments, the computer may generate a heatmap corresponding to the whole slide image. The heatmap may include tiles corresponding to the tiles of the whole slide image. An intensity value associated with each tile of the heatmap may be determined from the weighting value corresponding to the embedding feature vector of the corresponding tile of the whole slide image. In particular embodiments, the method further includes generating annotations for the whole slide image. The computer generates annotations for the whole slide image by identifying one or more weighting values satisfying a predetermined criteria, such as exceeding a threshold value, identifying one or more embedding feature vectors corresponding to the identified weighting values, and identifying one or more tiles corresponding to the identified embedding feature vectors. The annotations for the whole slide image may be provided for display in association with the whole slide image by marking the identified tiles or as an interactive overlay.

In particular embodiments, the computer may calculate a confidence score associated with the classification for the whole slide image from at least the weighting values and provide the confidence score for display in association with the classification for the whole slide image. In particular embodiments, the computer may identify, from the embedding feature vectors, weighting values, and slide embedding feature vector, derivative characteristics associated with the classification for the whole slide image. In particular embodiments, the computer may generate multiple classifications for multiple whole slide images, respectively and train one or more attention networks to predict weighting values associated with one or more conditions, respectively, using the classifications as a ground truth associated with the whole slide images. In particular embodiments, the whole slide image is received from a user device and the method includes providing the classification for the whole slide image to the user device for display. In particular embodiments, the whole slide image is received from a digital pathology image generation system communicatively coupled with a digital pathology image processing system that performs the method.

The embodiments disclosed herein are only examples, and the scope of this disclosure is not limited to them. Particular embodiments may include all, some, or none of the components, elements, features, functions, operations, or steps of the embodiments disclosed herein. Embodiments according to the invention are in particular disclosed in the attached claims directed to a method, a storage medium, a system and a computer program product, wherein any feature mentioned in one claim category, e.g., method, may be claimed in another claim category, e.g., system, as well. The dependencies or references back in the attached claims are chosen for formal reasons only. However any subject matter resulting from a deliberate reference back to any previous claims (in particular multiple dependencies) may be claimed as well, so that any combination of claims and the features thereof are disclosed and may be claimed regardless of the dependencies chosen in the attached claims. The subject-matter which may be claimed includes not only the combinations of features as set out in the attached claims but also any other combination of features in the claims, wherein each feature mentioned in the claims may be combined with any other feature or combination of other features in the claims. Furthermore, any of the embodiments and features described or depicted herein may be claimed in a separate claim and/or in any combination with any embodiment or feature described or depicted herein or with any of the features of the attached claims.

### BRIEF DESCRIPTION OF THE DRAWINGS

FIGS. 1A-1B illustrate an example embodiment of digital pathology image classification using multiple-instance learning.

FIG. 2 illustrates an example digital pathology image processing system and digital pathology image generation system.

FIG. 3 illustrates an example fully-connected attention network.

FIGS. 4A-4K illustrate example tile-based heatmaps of whole slide images.

FIG. 5A-5B illustrate example annotated whole slide images.

FIGS. 6A-6D illustrate an example embodiment of training an attention-based network and classification network for digital pathology images.

FIG. 7 illustrates and example method for digital pathology image classification.

FIG. 8 illustrates an example computer system.

### DESCRIPTION OF EXAMPLE EMBODIMENTS

As described herein, WSI are extremely large format digital images that may result from digitization of physical slides into high-resolution image files or may be output directly by medical scanning devices. WSI are typically preserved in the highest possible resolution format because of the nature of the images being captured and to avoid the misdiagnosis of tissue depicted in the WSI because of artifacts that ordinarily result from image compression and manipulation. WSI often include orders of magnitude larger numbers of pixels than typical digital images, and may include resolutions of 100,000 pixels by 100,000 pixels (e.g., 10,000 megapixels) or greater.

Analysis of WSI is a labor-intensive process that requires highly specialized individuals with the knowledge and dexterity to the review the WSI, recognize and identify abnormalities, classify the abnormalities, label the WSI, and potentially render diagnosis of the tissue. Additionally, because WSI are used for a wide array of tissue types, persons with the knowledge and skill to identify abnormalities must be further specialized in order to provide accurate analysis and diagnosis. Tissue abnormalities that may be detected from a WSI include, by way of example only and not limitation, inflammation, pigmentation, degeneration, anisokaryosis, hypertrophy, mitotic increase, mononuclear cell infiltration, inflammatory cell infiltration, inflammatory cell foci, decreased glycogen, glycogen accumulation (diffuse or concentrated), extramedullary myelopoiesis, extramedullary hematopoiesis, extramedullary erythropoiesis, single-cell necrosis, diffuse necrosis, marked necrosis, coagulative necrosis, apoptosis, karyomegaly, peribiliary, increased cellularity, glycogen deposits, lipid deposits, microgranuloma, congestion, Kupffer cell pigmentation, increased hemosiderin, histiocytosis, hyperplasia, or vacuolation, among many others. Therefore, because of the labor- and knowledge-intensive nature of the work, WSIs are considered candidate for automation certain functions. However, the large size of WSIs renders typical techniques ineffective, slow, and expensive. It is not practical to perform standard image recognition and deep learning techniques, which require analysis of multiple rounds of many samples of WSIs to increase accuracy. The techniques described herein are directed to solving the problem of automating feature recognition in WSI and enable the development of novel data analysis and presentation techniques that previously could not be performed with WSI due to the well-documented limitations.

The systems disclosed herein may efficiently generate training data for feature recognition based on standard WSI labels. Additionally, the present systems may identify whether a WSI contains abnormalities and where in the WSI the abnormalities are located.

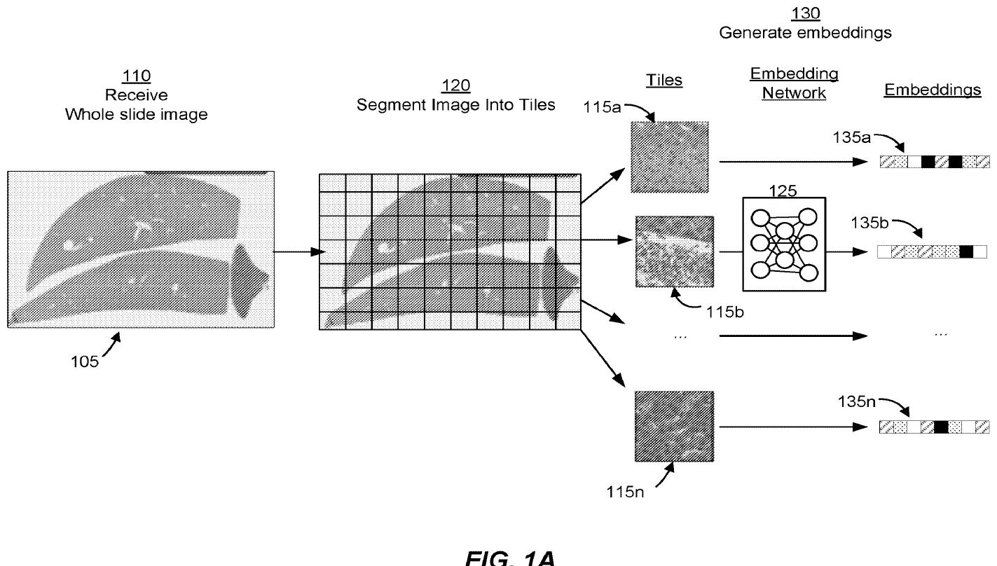

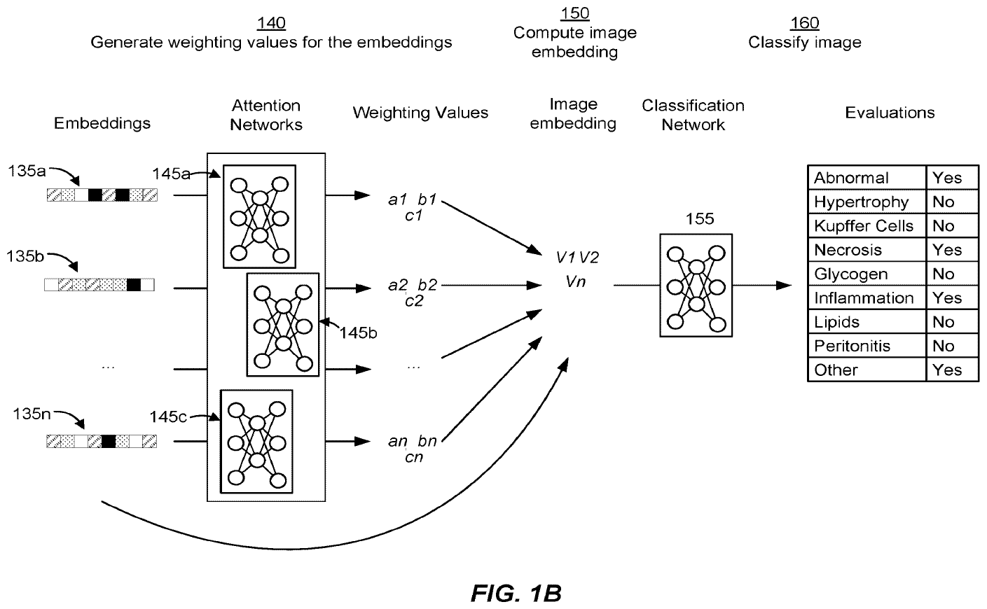

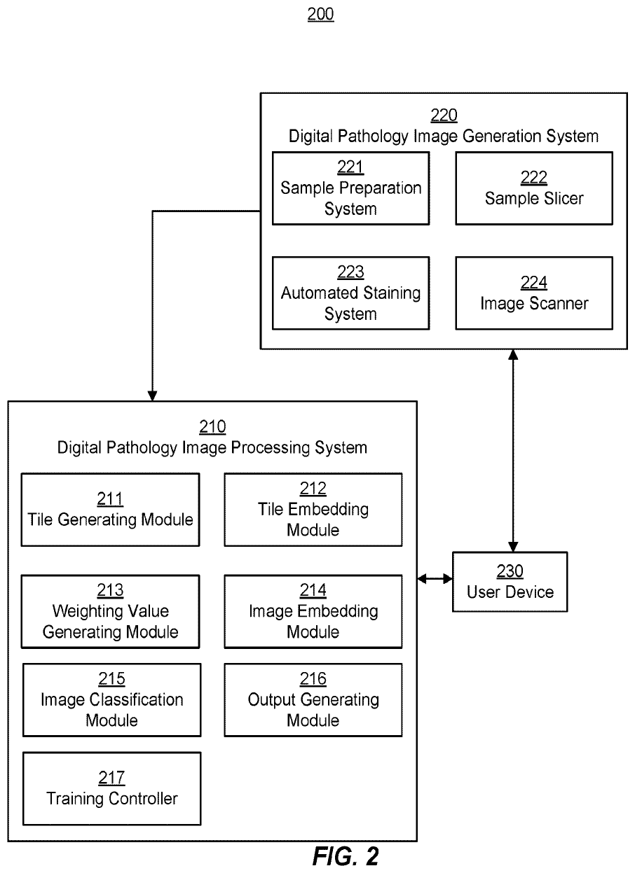

FIGS. 1A-1B illustrate an example process 100 for classifying whole slide images (WSI) using multiple-instance learning. FIG. 2 illustrates a network 200 of interacting computer systems that may be used, as described herein, for classifying whole slide images using neural networks and attention-based techniques according to some embodiments of the present disclosure. As illustrated in FIG. 1A, at 110, a digital pathology image processing system 210 receives a whole slide image 105. As an example only and by way of limitation, the digital pathology image processing system 210 may receive the whole slide image 105 from a digital pathology image generation system 220 or one or more components thereof. As another example, the digital pathology image processing system 210 may receive the whole slide image 105 from one or more user devices 230. User device 230 may be a computer used by a pathologist or clinician connected via one or more networks to the digital pathology image processing system 210. The user of the user device 230 may use the user device 230 to upload the whole slide image 105 or to direct one or more other devices to provide the whole slide image 105 to the digital pathology image processing system 210.

At 120 the digital pathology image processing system 210, for example using a tile-generating module 211, segments the whole slide image 105 into a plurality of tiles 115a, 115b, . . . 115n.

At 130 the digital pathology image processing system 210, for example using a tile embedding module 212, generates embeddings for each tile of the plurality of tiles using an embedding network 125. As an example, for tile 115a, the tile embedding module 212 generates a corresponding embedding 135a, for tile 115b, the tile embedding module 212 generates a corresponding embedding 135b, and for tile 115n, the tile embedding module 212 generates a corresponding embedding 135n. As described herein, the embeddings may include unique representations of the tiles that preserve some information about the content or context of the tiles. The tile embeddings may also be derived from a translation of the tiles into a corresponding tile embedding space, where distinct within the tile embedding space correlates to similarity of the tiles. For example, tiles that depict similar subject matter or have similar visual features will be positioned closer in the embedding space than tiles that depict different subject matter or have dissimilar visual features. The tile embeddings may be represented as feature vectors.

As illustrated in FIG. 1B, at 140 the digital pathology image processing system 210, for example using a weighting value generating module 213, generates weighting values for each of the embeddings 135a, 135b, . . . 135n. For example, the weighting value generating module 213 generates weighting values a₁, b₁, and c₁ for embedding 135a, generates weighting values a₂, b₂, and c₃, for embedding 135b, and generates weighting values aₙ, bₙ, and cₙ for the embedding 135n. To generate the weighting values, the weighting value generating module 213 may use multiple attention networks 145a, 145b, . . . 145c to generate attention scores for the embeddings, described herein, before the embeddings and subsequently normalized for use as weighting values. In certain embodiments, each attention network generates a weighting value for each embedding, such that the number of weighting values generated for each embedding is equivalent to the number of attention networks used by the weighting value generating module 213.

At 150, the digital pathology image processing system 210, for example using an image embedding module 214, computes image embeddings V₁, V₂, . . . Vₙ for the whole slide image 105 by combining the tile embeddings in a weighted combination, using the weighting values generated for each embedding to weight the respective embedding. In some embodiments, multiple image embeddings V₁, V₂, . . . Vₙ may be generated, for example one image embedding for each attention network 145a, 145b, 145c. The image embedding Vₙ may be calculated as a weighted combination, Vₙ=α₁+α₂+ . . . +αₙ. In some embodiments, a single image embedding may be generated using all of the weighting values (e.g., weighting values from all of the attention networks).

At 160, the digital pathology image processing system 210, for example using an image classification module 215, classifies the whole slide image 105 using the image embeddings V₁, V₂, . . . Vₙ. In some embodiments, the image classification module 215 uses an classification network 155 to generate the classifications. The classifications are then presented as evaluations of the whole slide image, where the evaluations are equivalent to predictions of one or more conditions present in the whole slide image. For example, the evaluations may include a determination that the whole slide image depicts normal biological conditions or contains diagnosable biological abnormalities. Diagnosable biological abnormalities may include abnormalities associated with hypertrophy (e.g., hepatocyte hypertrophy, Kupffer cell hypertrophy, etc.), Kupffer cells (e.g., Kupffer cell pigmentation, Kupffer cell hypertrophy, etc.), necrosis (e.g., diffuse, focal, coagulative, etc.), glycogen (e.g., glycogen depletion, glycogen deposits, etc.), inflammation, lipids (e.g., lipid depletion, lipid deposits, etc.), peritonitis, and other conditions. As another example, the evaluations may include a determination that indications of one or more conditions are present in the whole slide image. The evaluations may be provided to users or operators of the digital pathology image processing system 210 for review. The evaluations may also be provided to one or more user devices 230.

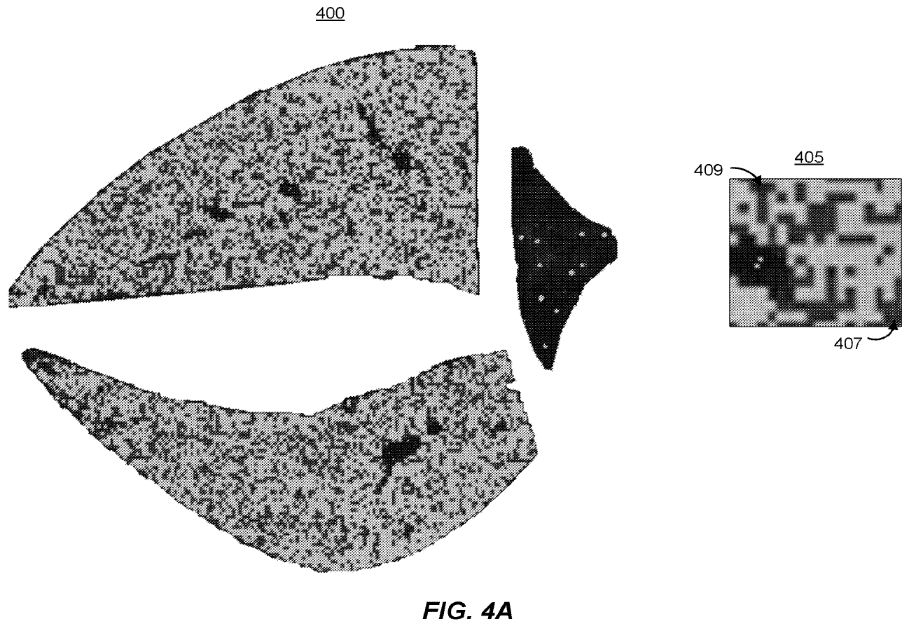

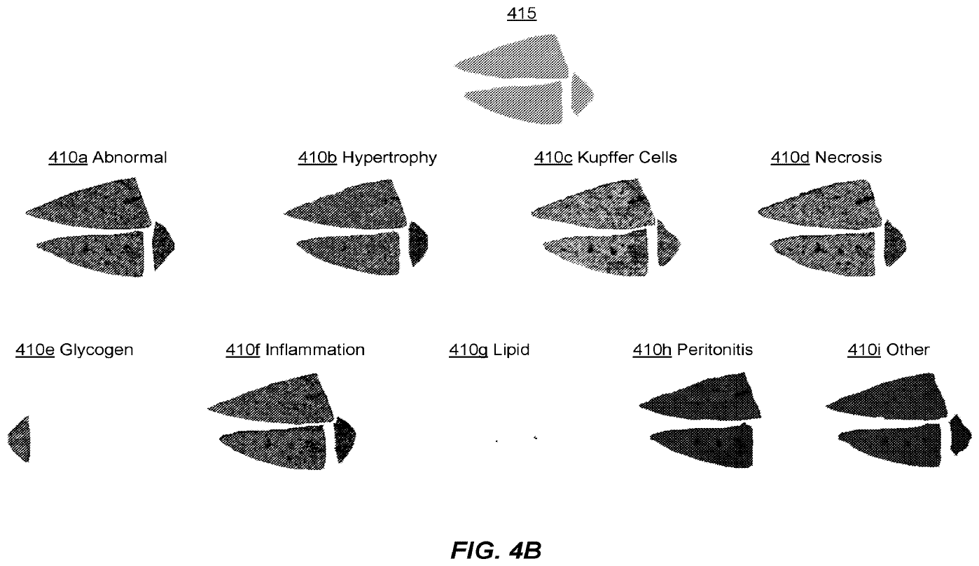

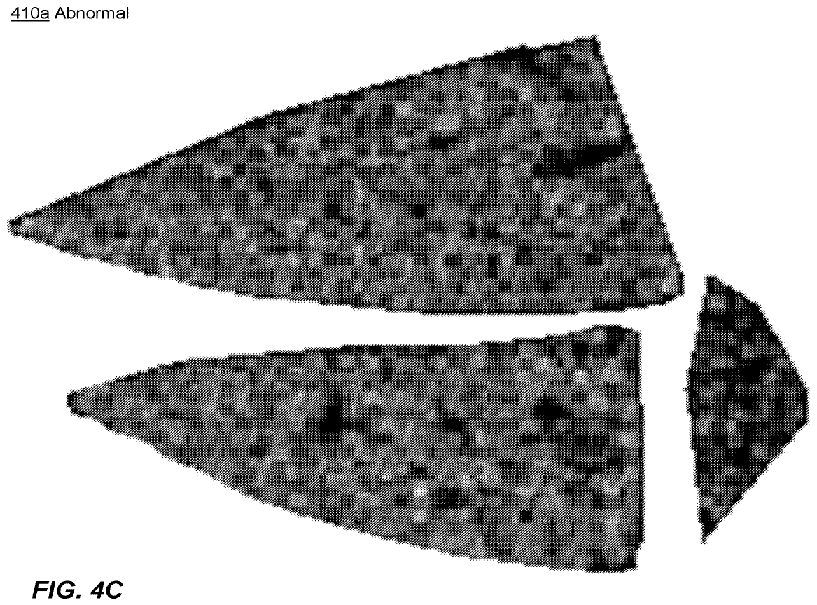

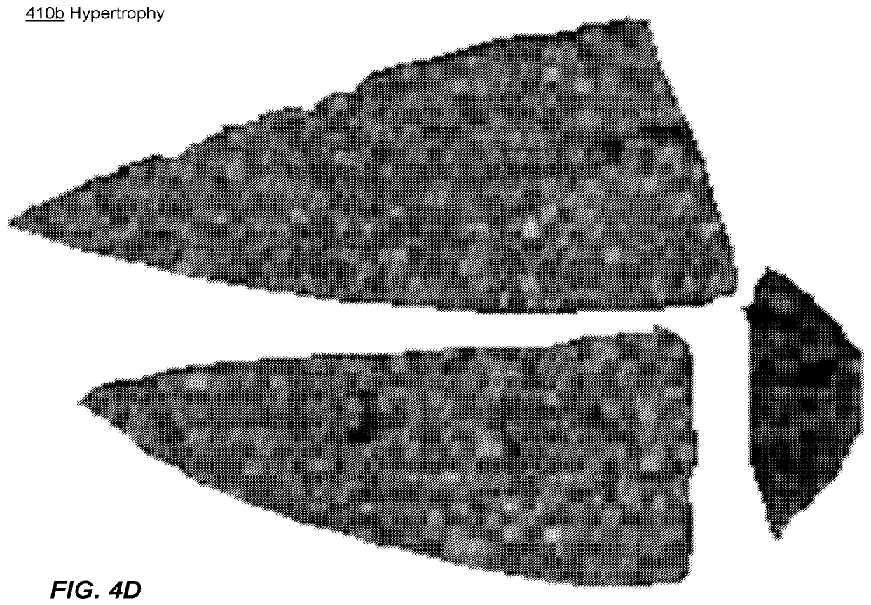

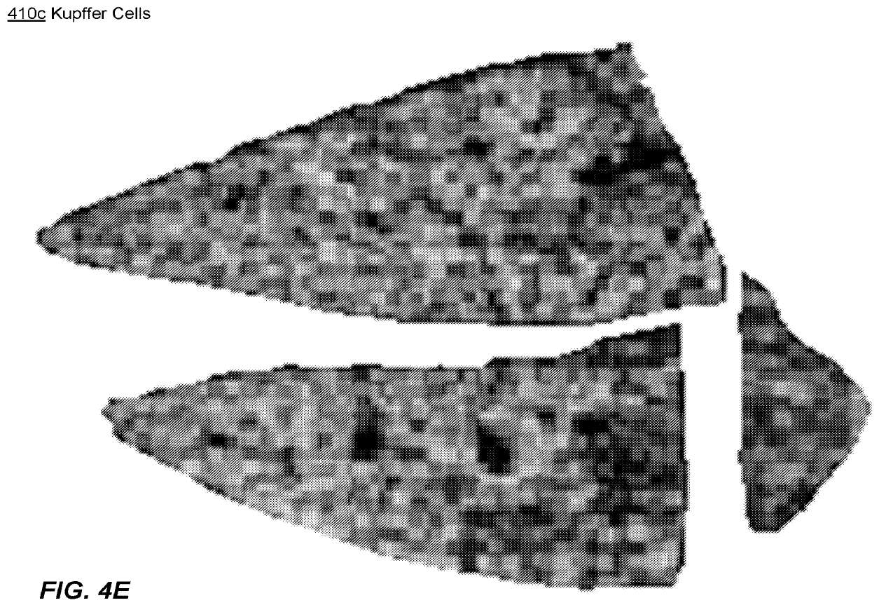

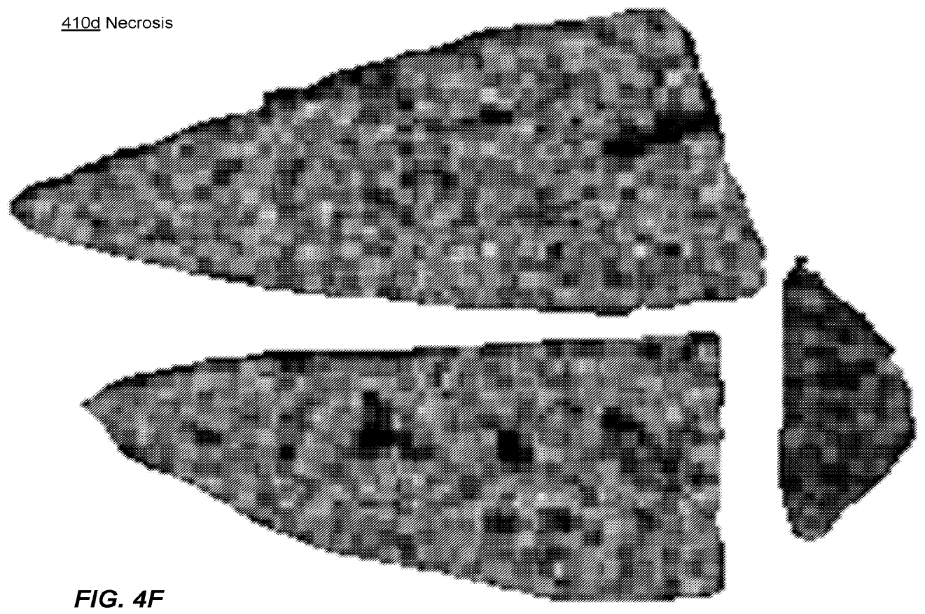

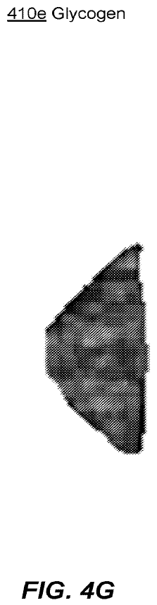

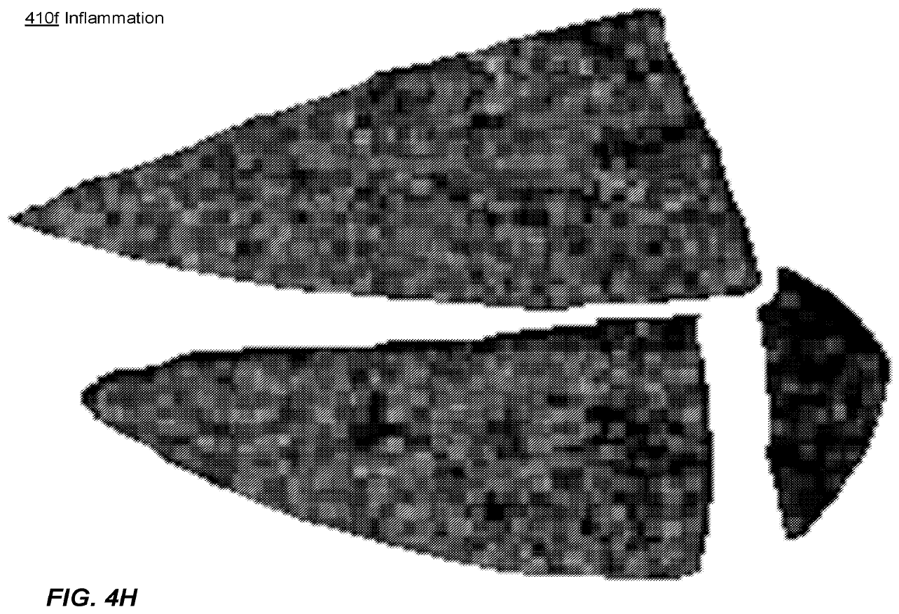

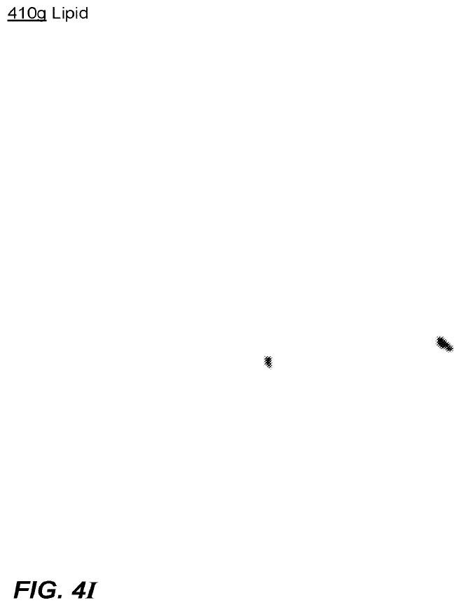

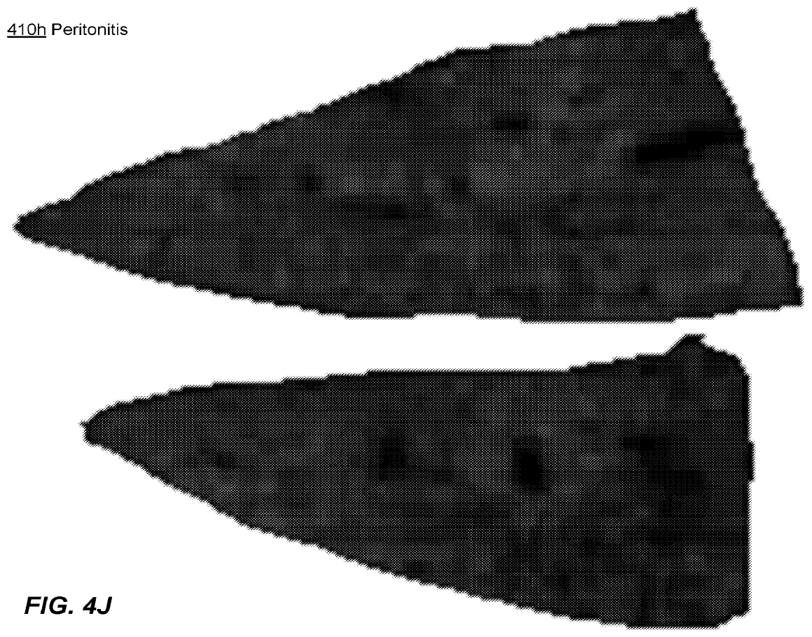

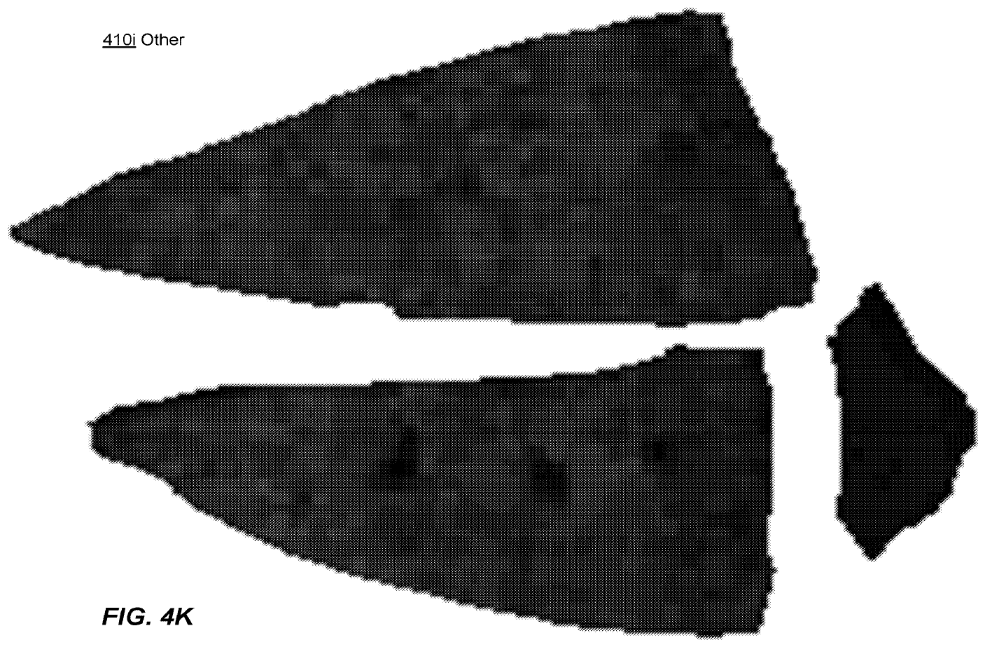

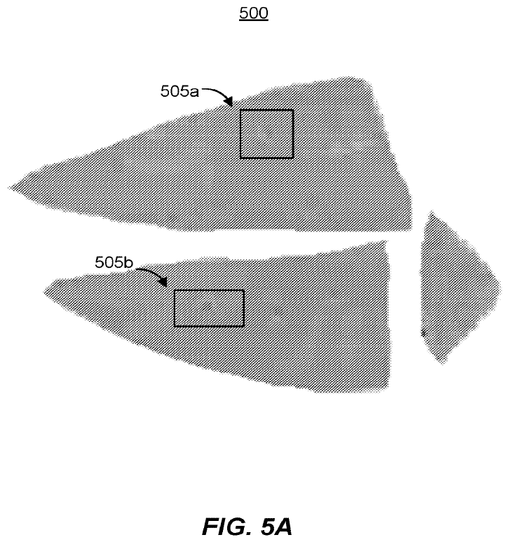

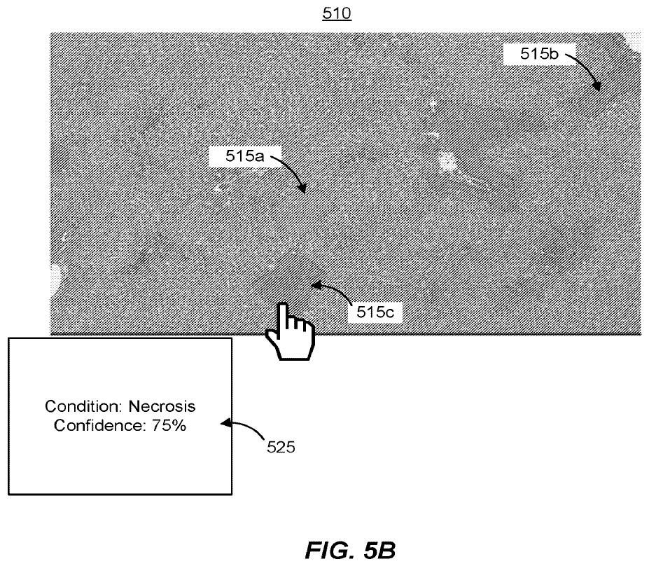

As discussed herein, the output from the digital pathology image processing system 210 may be provided in a number of forms, including a simple recitation of the evaluations made by the digital pathology image processing system. More advanced output may also be provided. As an example, the digital pathology image processing system 210 may generate “heatmaps” of the whole slide image where the value of each tile of the heatmap is correlated to the value of one or more of the weighting values generated by the attention networks. Example heatmaps are illustrated in FIGS. 4A and 4B. The digital pathology image processing system 210 may further generate an annotation overlay for the image that groups and identifies regions of the image that are relevant to a particular category or that are otherwise suggested for review by the user of a user device 230. Example annotation overlays are illustrated in FIGS. 5A and 5B.

FIG. 2 illustrates a network 200 of interacting computer systems that may be used, as described herein, for classifying whole slide images using neural networks and attention-based techniques according to some embodiments of the present disclosure.

A digital pathology image generation system 220 may generate one or more digital pathology images, including, but not limited to whole slide images, corresponding to a particular sample. For example, an image generated by digital pathology image generation system 220 may include a stained section of a biopsy sample. As another example, an image generated by digital pathology image generation system 220 may include a slide image (e.g., a blood film) of a liquid sample. As another example, an image generated by digital pathology image generation system 220 may include fluorescence microscopy such as a slide image depicting fluorescence in situ hybridization (FISH) after a fluorescent probe has been bound to a target DNA or RNA sequence.

Some types of samples (e.g., biopsies, solid samples and/or samples including tissue) may be processed by a sample preparation system 221 to fix and/or embed the sample. Sample preparation system 221 may facilitate infiltrating the sample with a fixating agent (e.g., liquid fixing agent, such as a formaldehyde solution) and/or embedding substance (e.g., a histological wax). For example, a sample fixation sub-system may fix a sample by exposing the sample to a fixating agent for at least a threshold amount of time (e.g., at least 3 hours, at least 6 hours, or at least 12 hours). A dehydration sub-system may dehydrate the sample (e.g., by exposing the fixed sample and/or a portion of the fixed sample to one or more ethanol solutions) and potentially clear the dehydrated sample using a clearing intermediate agent (e.g., that includes ethanol and a histological wax). A sample embedding sub-system may infiltrate the sample (e.g., one or more times for corresponding predefined time periods) with a heated (e.g., and thus liquid) histological wax. The histological wax may include a paraffin wax and potentially one or more resins (e.g., styrene or polyethylene). The sample and wax may then be cooled, and the wax-infiltrated sample may then be blocked out.

A sample slicer 222 may receive the fixed and embedded sample and may produce a set of sections. Sample slicer 222 may expose the fixed and embedded sample to cool or cold temperatures. Sample slicer 222 may then cut the chilled sample (or a trimmed version thereof) to produce a set of sections. Each section may have a thickness that is (for example) less than 100 μm, less than 50 μm, less than 10 μm or less than 5 μm. Each section may have a thickness that is (for example) greater than 0.1 μm, greater than 1 μm, greater than 2 μm or greater than 4 μm. The cutting of the chilled sample may be performed in a warm water bath (e.g., at a temperature of at least 30° C., at least 35° C. or at least 40° C.).

An automated staining system 223 may facilitate staining one or more of the sample sections by exposing each section to one or more staining agents. Each section may be exposed to a predefined volume of staining agent for a predefined period of time. In some instances, a single section is concurrently or sequentially exposed to multiple staining agents.

Each of one or more stained sections may be presented to an image scanner 224, which may capture a digital image of the section. Image scanner 224 may include a microscope camera. The image scanner 224 may capture the digital image at multiple levels of magnification (e.g., using a 10× objective, 20× objective, 40× objective, etc.). Manipulation of the image may be used to capture a selected portion of the sample at the desired range of magnifications. Image scanner 224 may further capture annotations and/or morphometrics identified by a human operator. In some instances, a section is returned to automated staining system 223 after one or more images are captured, such that the section may be washed, exposed to one or more other stains and imaged again. When multiple stains are used, the stains may be selected to have different color profiles, such that a first region of an image corresponding to a first section portion that absorbed a large amount of a first stain may be distinguished from a second region of the image (or a different image) corresponding to a second section portion that absorbed a large amount of a second stain.

It will be appreciated that one or more components of digital pathology image generation system 220 can, in some instances, operate in connection with human operators. For example, human operators may move the sample across various sub-systems (e.g., of sample preparation system 221 or of digital pathology image generation system 220) and/or initiate or terminate operation of one or more sub-systems, systems or components of digital pathology image generation system 220. As another example, part or all of one or more components of digital pathology image generation system (e.g., one or more subsystems of the sample preparation system 221) may be partly or entirely replaced with actions of a human operator.

Further, it will be appreciated that, while various described and depicted functions and components of digital pathology image generation system 220 pertain to processing of a solid and/or biopsy sample, other embodiments may relate to a liquid sample (e.g., a blood sample). For example, digital pathology image generation system 220 may receive a liquid-sample (e.g., blood or urine) slide that includes a base slide, smeared liquid sample and cover. Image scanner 224 may then capture an image of the sample slide. Further embodiments of the digital pathology image generation system 220 may relate to capturing images of samples using advancing imaging techniques, such as FISH, described herein. For example, once a florescent probe has been introduced to a sample and allowed to bind to a target sequence appropriate imaging may be used to capture images of the sample for further analysis.

A given sample may be associated with one or more users (e.g., one or more physicians, laboratory technicians and/or medical providers) during processing and imaging. An associated user may include, by way of example and not of limitation, a person who ordered a test or biopsy that produced a sample being imaged, a person with permission to receive results of a test or biopsy, or a person who conducted analysis of the test or biopsy sample, among others. For example, a user may correspond to a physician, a pathologist, a clinician, or a subject. A user may use one or one user devices 230 to submit one or more requests (e.g., that identify a subject) that a sample be processed by digital pathology image generation system 220 and that a resulting image be processed by a digital pathology image processing system 210.

Digital pathology image generation system 220 may transmit an image produced by image scanner 224 back to user device 230. User device 230 then communicates with the digital pathology image processing system 210 to initiate automated processing of the image. In some instances, digital pathology image generation system 220 provides an image produced by image scanner 224 to the digital pathology image processing system 210 directly, e.g. at the direction of the user of a user device 230. Although not illustrated, other intermediary devices (e.g., data stores of a server connected to the digital pathology image generation system 220 or digital pathology image processing system 210) may also be used. Additionally, for the sake of simplicity only one digital pathology image processing system 210, image generating system 220, and user device 230 is illustrated in the network 200. This disclosure anticipates the use of one or more of each type of system and component thereof without necessarily deviating from the teachings of this disclosure.

The network 200 and associated systems shown in FIG. 2 may be used in a variety of contexts where scanning and evaluation of digital pathology images, such as whole slide images, are an essential component of the work. As an example, the network 200 may be associated with a clinical environment, where a user is evaluating the sample for possible diagnostic purposes. The user may review the image using the user device 230 prior to providing the image to the digital pathology image processing system 210. The user may provide additional information to the digital pathology image processing system 210 that may be used to guide or direct the analysis of the image by the digital pathology image processing system 210. For example, the user may provide a prospective diagnosis or preliminary assessment of features within the scan. The user may also provide additional context, such as the type of tissue being reviewed. As another example, the network 200 may be associated with a laboratory environment were tissues are being examined, for example, to determine the efficacy or potential side effects of a drug. In this context, it may be commonplace for multiple types of tissues to be submitted for review to determine the effects on the whole body of said drug. This may present a particular challenge to human scan reviewers, who may need to determine the various contexts of the images, which may be highly dependent on the type of tissue being imaged. These contexts may optionally be provided to the digital pathology image processing system 210.

Digital pathology image processing system 210 may process digital pathology images, including whole slide images, to classify the digital pathology images and generate annotations for the digital pathology images and related output. A tile generating module 211 may define a set of tiles for each digital pathology image. To define the set of tiles, the tile generating module 211 may segment the digital pathology image into the set of tiles. As embodied herein, the tiles may be non-overlapping (e.g., each tile includes pixels of the image not included in any other tile) or overlapping (e.g., each tile includes some portion of pixels of the image that are included in at least one other tile). Features such as whether or not tiles overlap, in addition to the size of each tile and the stride of the window (e.g., the image distance or pixels between a tile and a subsequent tile) may increase or decrease the data set for analysis, with more tiles (e.g., through overlapping or smaller tiles) increasing the potential resolution of eventual output and visualizations. In some instances, tile generating module 211 defines a set of tiles for an image where each tile is of a predefined size and/or an offset between tiles is predefined. Furthermore, the tile generating module 211 may create multiple sets of tiles of varying size, overlap, step size, etc., for each image. In some embodiments, the digital pathology image itself may contain tile overlap, which may result from the imaging technique. Even segmentation without tile overlapping may be a preferable solution to balance tile processing requirements and avoid influencing the embedding generation and weighting value generation discussed herein. A tile size or tile offset may be determined, for example, by calculating one or more performance metrics (e.g., precision, recall, accuracy, and/or error) for each size/offset and by selecting a tile size and/or offset associated with one or more performance metrics above a predetermined threshold and/or associated with one or more optimal (e.g., high precision, highest recall, highest accuracy, and/or lowest error) performance metric(s). The tile generating module 211 may further define a tile size depending on the type of abnormality being detected. For example, the tile generating module 211 may be configured with awareness of the type(s) of tissue abnormalities that the digital pathology image processing system 210 will be searching for and may customize the tile size according to the tissue abnormalities to optimize detection. For example, the image generating module 211 may determine that, when the tissue abnormalities include searching for inflammation or necrosis in lung tissue, the tile size should be reduced to increase the scanning rate, while when the tissue abnormalities include abnormalities with Kupffer cells in liver tissues, the tile size should be increased to increase the opportunities for the digital pathology image processing system 210 to analyze the Kupffer cells holistically. In some instances, tile generating module 211 defines a set of tiles where a number of tiles in the set, size of the tiles of the set, resolution of the tiles for the set, or other related properties, for each image is defined and held constant for each of one or more images.

As embodied herein, the tile generating module 211 may further define the set of tiles for each digital pathology image along one or more color channels or color combinations. As an example, digital pathology images received by digital pathology image processing system 210 may include large-format multi-color channel images having pixel color values for each pixel of the image specified for one of several color channels. Example color specifications or color spaces that may be used include the RGB, CMYK, HSL, HSV, or HSB color specifications. The set of tiles may be defined based on segmenting the color channels and/or generating a brightness map or greyscale equivalent of each tile. For example, for each segment of an image, the tile generating module 211 may provide a red tile, blue tile, green tile, and/or brightness tile, or the equivalent for the color specification used. As explained herein, segmenting the digital pathology images based on segments of the image and/or color values of the segments may improve the accuracy and recognition rates of the networks used to generating embeddings for the tiles and image and to produce classifications of the image. Additionally, the digital pathology image processing system 210, e.g., using tile generating module 211, may convert between color specifications and/or prepare copies of the tiles using multiple color specifications. Color specification conversions may be selected based on a desired type of image augmentation (e.g., accentuating or boosting particular color channels, saturation levels, brightness levels, etc.). Color specification conversions may also be selected to improve compatibility between digital pathology image generation systems 220 and the digital pathology image processing system 210. For example, a particular image scanning component may provide output in the HSL color specification and the models used in the digital pathology image processing system 210, as described herein, may be trained using RGB images. Converting the tiles to the compatible color specification may ensure the tiles may still be analyzed. Additionally, the digital pathology image processing system may up-sample or down-sample images that are provided in particular color depth (e.g., 8-bit, 16-bit, etc.) to be usable by the digital pathology image processing system. Furthermore, the digital pathology image processing system 210 may cause tiles to be converted according to the type of image that has been captured (e.g., fluorescent images may include greater detail on color intensity or a wider range of colors).

As described herein, a tile embedding module 212 may generate an embedding (e.g., 135a, 135b, . . . 135n) for each tile in a corresponding embedding space. The embedding may be represented by the digital pathology image processing system 210 as a feature vector for the tile. The tile embedding module 212 may use a neural network (e.g., a convolutional neural network) to generate a feature vector that represents each tile of the image. In particular embodiments, the tile embedding neural network may be based on the ResNet image network trained on a dataset based on natural (e.g., non-medical) images, such as the ImageNet dataset. By using a non-specialized tile embedding network, the tile embedding module 212 may leverage known advances in efficiently processing images to generating embeddings. Furthermore, using a natural image dataset allows the embedding neural network to learn to discern differences between tile segments on a holistic level.

In other embodiments, the tile embedding network used by the tile embedding module 212 may be an embedding network customized to handle large numbers of tiles of large format images, such as digital pathology whole slide images. Additionally, the tile embedding network used by the tile embedding module 212 may be trained using a custom dataset. For example, the tile embedding network may be trained using a variety of samples of whole slide images or even trained using samples relevant to the subject matter for which the embedding network will be generating embeddings (e.g., scans of particular tissue types). Training the tile embedding network using specialized or customized sets of images may allow the tile embedding network to identify finer differences between tiles which may result in more detailed and accurate distances between tiles in the embedding space at the cost of additional time to acquire the images and the computational and economic cost of training multiple tile generating networks for use by the tile embedding module 212. The tile embedding module 212 may select from a library of tile embedding networks based on the type of images being processed by the digital pathology image processing system 210.

As described herein, tile embeddings (e.g., 135a, 135b, . . . 135n) may be generated from a deep learning neural network using visual features of the tiles. Tile embeddings may be further generated from contextual information associated with the tiles or from the content shown in the tile. For example, a tile embedding may include one or more features that indicate and/or correspond to a size of depicted objects (e.g., sizes of depicted cells or aberrations) and/or density of depicted objects (e.g., a density of depicted cells or aberrations). Size and density may be measured absolutely (e.g., width expressed in pixels or converted from pixels to nanometers) or relative to other tiles from the same digital pathology image, from a class of digital pathology images (e.g., produced using similar techniques or by a single digital pathology image generation system or scanner), or from a related family of digital pathology images. Furthermore, tiles may be classified prior to the tile embedding module 212 generating embeddings for the tiles such that the tile embedding module 212 considers the classification when preparing the embeddings.

For consistency, the tile embedding module 212 produces embeddings of a predefined size (e.g., vectors of 512 items, vectors of 2048 bytes, etc.). The tile embedding module 212 may produce embeddings of various and arbitrary sizes. The time embedding module 212 may adjust the sizes of the embeddings based on user direction or may be selected, for example, to optimize computation efficiency, accuracy, or other parameters. In particular embodiments, the embedding size may be based on the limitations or specifications of the deep learning neural network that generated the embeddings. Larger embedding sizes may be used to increase the amount of information captured in the embedding and improve the quality and accuracy of results, while smaller embedding sizes may be used to improve computational efficiency.

A weighting value generating module 213 may generate a weighting value for each tile that will be used in association with the tile and the corresponding embedding. As described herein, the weighting value may be an attention score generated by a neural network that receives tile embeddings as input and generates attention scores as output, also referred to as an attention neural network or simply an attention network. For example, the attention score may be defined to be and/or interpreted to be an extent to which a given tile is predictive of a specific output. A tiles, or tile embedding, with a high attention score relative to other tiles in a set may be said to have been identified by the attention network has having a high influence in the classification of the digital pathology image. For example, the attention network may learn that certain features in the tile or tile embedding are highly relevant to a digital pathology image being classified as normal or abnormal or as indicating inflammation or necrosis. The weighting value generating module 213 may use multiple attention networks as needed, including at least one for each class of output that the digital pathology image processing system 210 may detect. As an example, the weighting value generating module 213 may use one or more attention networks that have been trained, as described herein, to determine the key instances of tiles associated with each of multiple conditions that are detectable in the digital pathology image. As an example only and not by way of limitation, the weighting value generating module 213 may include networks trained to detect particular diagnoses which may be grouped according to the similarities or likelihood of usefulness to an end user. For example, the networks may be trained to detect conditions includes hypertrophy (e.g., hepatocyte hypertrophy, Kupffer cell hypertrophy, etc.), Kupffer cells (e.g., Kupffer cell pigmentation, Kupffer cell hypertrophy, etc.), necrosis (e.g., diffuse, focal, coagulative, etc.), glycogen (e.g., glycogen depletion, glycogen deposits, etc.), inflammation, lipids (e.g., lipid depletion, lipid deposits, etc.), peritonitis, and other conditions detectable in a digital pathology image. Additionally, the weighting value generating module 213 may include an attention network trained to determine abnormalities in the tiles of the digital pathology images and assign an overall weighting value for abnormal versus normal.

While some embodiments may include each given tile having a respective attention score, other embodiments may include attentions cores that correspond to regions of an image that may include or comprise one or more tiles or portion of tiles. For example, such image regions may extend beyond the borders of a single tile or may have a perimeter that is smaller than that of a single tile. Attention scores may result from processing of image related details (e.g., intensities and/or color values) within the tile or image region. Contextual information for the tile, such as the position of the tile within the digital pathology image, may also be used by the attention network to generate the attention score. The attention network receives a series of embeddings (e.g., vector representations) that correspond to a set of pixel intensities or to a position within an embedding space. The attention network may include, for example, a feed-forward network, perceptron network (e.g., a multilayer perceptron), and/or a network having one or more fully connected layers. The neural network may further include a convolutional neural network and one or more additional layers (e.g., a fully connected layer).

An image embedding module 214 generates an embedding for the digital pathology image (e.g., the whole slide image) using the tile embeddings (e.g., 135a, 135b, . . . 135n) and the weighting values. The image embedding may take the form of another feature vector to represent the image. As embodied herein, the image embedding may result from a combination of the tile embeddings where the weighting values generated by the weighting value generating module 213 are used to weight the tile embeddings. In other words, the image embedding may be the result of a weighted combination of the tile embeddings according to the attentions score from each attention network. In generating the image embedding, the image embedding module 214 may apply further transformations and/or normalizations to the tile embeddings (e.g., 135a, 135b, . . . 135n) and weighting values. Therefore, one or more image embeddings may be generated. In particular, the image embedding module 214 may generate one image embedding for each attention network (and thus each condition being evaluated). The image embedding module 214 may also generate one or more composite embeddings where embeddings and weighting values across attention networks are combined.

An image classification module 215 then processes the image embedding to determine which classifications should be applied to the digital pathology image. The image classification module 215 may include or use one or more classification networks 155 trained to classify a digital pathology image from the image embedding. For example, a single classification network 155 may be trained to identify and differentiate between classifications. In another example, one classification network 155 may be used for each classification or condition of interest, such that each classification network 155 determines that the image embedding is indicative of its subject classification or condition or not. The resulting classification(s) may be interpreted as evaluations of the digital pathology image and determinations that the digital pathology image includes indicators of one or more specified conditions. For example, the output of the image classification module 215 may include a series of binary yes or no determinations for a sequence of conditions. The output may be further organized as a vector composed of the yes or no determinations. The determinations may be augmented, for example, with a confidence score or interval representing the degree of confidence that the image classification module 215 or its component classification networks 155 have in a particular determination. For example, the image classification module 215 may indicate that the digital image is 85% likely to include abnormal cells, 80% likely to not be indicative of hypertrophy, 60% likely to be indicative of inflammation, etc. Additionally, the output of the classifier network(s) may include a set of scores associated with each potential classification. The image classification module 215 may then apply a normalizing function (e.g., softmax, averaging, etc.) to the scores before assessing the scores and assigning a confidence level. In this manner, the digital pathology image processing system 210 may automatically label for digital pathology images from the image embeddings, which are in turn based on tile embeddings and weighting values.

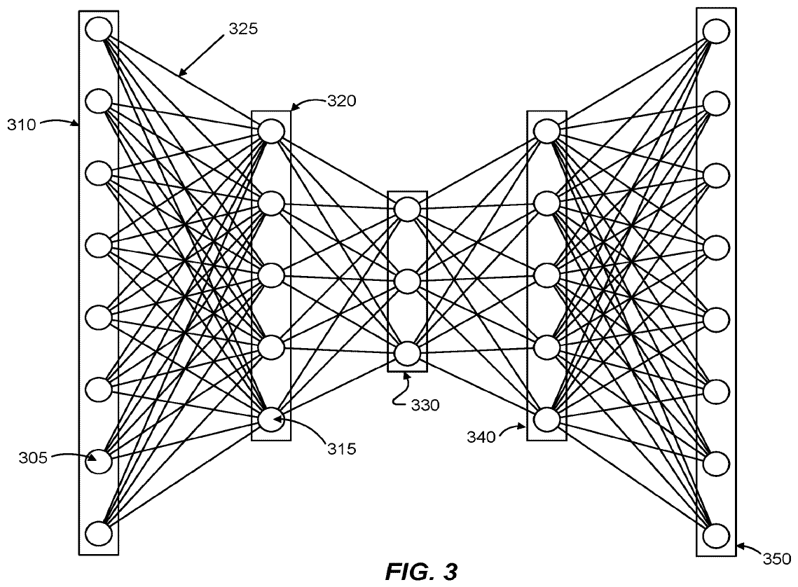

As described herein, the image embedding network, attention networks, and classification network may be artificial neural networks (“ANN”) designed and trained for a specific function. FIG. 3 illustrates an example ANN 300. An ANN may refer to a computational model comprising one or more nodes. An example ANN 300 includes an input layer 310, hidden layers 320, 330, 340, and an output layer 350. Each layer of the ANN 300 may include one or more nodes, such as a node 305 or a node 315. In particular embodiments, one or more nodes of an ANN may be connected to another node of the ANN. In a fully-connected ANN, each node of an ANN is connected to each node of the preceding and/or subsequent layers of the ANN. As an example and not by way of limitation, each node of the input layer 310 may be connected to each node of the hidden layer 320, each node of the hidden layer 320 may be connected to each node of hidden layer 330, and so on. In particular embodiments, one or more nodes is a bias node, which may be a node that is not connected to and does not receive input from any node in a previous layer. Although FIG. 3 depicts a particular ANN 300 with a particular number of layers, a particular number of nodes, and particular connections between nodes, this disclosure contemplates any suitable ANN with any suitable number of layers, any suitable number of nodes, and any suitable connections between nodes. As an example, FIG. 3 depicts a connection between each node of the input layer 310 and each node of the hidden layer 320, although in particular embodiments, one or more nodes of the input layer 310 is not connected to one or more nodes of the hidden layer 320 and the same applies for the remaining nodes and layers of the ANN 300.

ANNs used in particular embodiments may be a feedforward ANN with no cycles or loops and where communication between nodes flows in one direction beginning with the input layer and proceeding to successive layers. As an example, the input to each node of the hidden layer 320 may include the output of one or more nodes of the input layer 310. Similarly, the input to each node of the output layer 350 may include the output of nodes of the hidden layer 340. ANNs used in particular embodiments may be deep neural networks having least two hidden layers. ANNs used in particular embodiments may be deep residual networks, a feedforward ANN including hidden layers organized into residual blocks. The input into each residual block after the first residual block may be a function of the output of the previous residual block and the input of the previous residual block. As an example and not by way of limitation, the input into residual block N may be represented as F(x)+x, where F(x) is the output of residual block N−1, and x is the input into residual block N−1. Although this disclosure describes a particular ANN, this disclosure contemplates any suitable ANN.

In particular embodiments, each node of an ANN may include an activation function. The activation function of a node defines or describes the output of the node for a given input. In particular embodiments, the input to a node may be a singular input or may include a set of inputs. Example activation functions may include an identity function, a binary step function, a logistic function, or any other suitable function. Example activation functions for a node k may include the sigmoid function Fₖ(sₖ)=1/1+e^{-s}^{ₖ} the hyperbolic tangent function Fₖ(sₖ)=e^{s}k−e^{-s}^{ₖ}/e^{s}k+e^{-s}^{ₖ}, the rectifier Fₖ(sₖ)=max(0,sₖ), or any other suitable function Fₖ(sₖ), where sₖ is the input to node k.

The input of an activation function corresponding to a node may be weighted. Each node may generate output using a corresponding activation function based on weighted inputs. As embodied herein, each connection between nodes may be associated with a weight. For example, a connection 325 between the node 305 and the node 315 may have a weighting coefficient of 0.4, which indicates that the input of node 315 is 0.4 (the weighting coefficient) multiplied by the output of the node 305. More generally, the output y k of node k may be yₖ=Fₖ(sₖ), where Fₖ is the activation function corresponding to node k, sₖ=Σⱼ(wⱼₖxⱼ) is the input to node k, xⱼ is the output of a node j connected to node k, and wⱼₖ is the weighting coefficient between node j and node k. As embodied herein, the input to nodes of the input layer 310 may be based on a vector representing an object, also referred to as a vector representation of the object, an embedding of the object in a corresponding embedding space, or other suitable input. Although this disclosure describes particular inputs to and outputs of nodes, this disclosure contemplates any suitable inputs to and outputs of nodes in an ANN. Moreover, although this disclosure describes particular connections and weights between nodes, this disclosure contemplates any suitable connections and weights between nodes.

In particular embodiments, an ANN 300 may be trained using training data. As an example and not by way of limitation, training data may include inputs to the ANN 300 and an expected output, such as a ground truth value corresponding to the input. For example, training data may include one or more vectors representing a training object and an expected label for the training object. Training typically occurs with multiple training objects simultaneously or in succession. Training an ANN may include modifying the weights associated with the connections between nodes of the ANN by optimizing an objective function. As an example and not by way of limitation, a training method may be used to backpropagate an error value. The error value may be measured as a distance between each vector representing a training object, for example, using a cost function that minimizes error or a value derived from the error, such as a sum-of-squares error. Example training methods include, but are not limited to the conjugate gradient method, the gradient descent method, the stochastic gradient descent, etc. In particular embodiments, an ANN may be trained using a dropout technique in which one or more nodes are temporarily omitted while training such that they receive no input or produce no output. For each training object, one or more nodes of the ANN have a probability of being omitted. The nodes that are omitted for a particular training object may differ from nodes omitted for other training objects. Although this disclosure describes training an ANN in a particular manner, this disclosure contemplates training an ANN in any suitable manner.

The weighting value generating module 213 may further apply normalizing functions to the attention scores associated with each embedding for the tiles. The normalizing functions may be used to normalize weighting values (e.g., attention scores) across the tiles. For example, one normalizing function that may be applied is the softmax function:

$${o ( \vec{z} )}_{i} = \frac{{e}^{{\vec{z}}_{i}}}{{\sum }_{j = 1}^{K} {e}^{{\vec{z}}_{j}}}$$

where \vec{z} is an input vector, e^{z}^{ᵢ} is the standard exponential function for the input vector, K is the number of classes in the multi-class classifier, e^{z}^{ⱼ} is the standard exponential function for an output vector. The softmax function applies the standard exponential function to each element of the input vector and normalizes the values by dividing the sum of all the exponentials. The normalization ensures that the sum of the components of the output vector is equal to 1. The normalizing function may include modifications to the softmax function (e.g., using a different exponential function) or may use alternatives to the softmax function entirely.

An output generating module 216 of the digital pathology image processing system 210 may use the digital pathology image, tiles, tile embeddings, weighting values, image embedding, and classifications to generate output corresponding to the digital pathology image received as input. As described herein, in addition to labels and annotations for the digital pathology image, the output may include a variety of visualizations and interactive graphics. In many embodiments, the output will be provided to the user device 230 for display, but in certain embodiments the output may be access directly from the digital pathology image processing system 210.

In one embodiment, the output for a given digital pathology image may include a so-called heatmap that identifies and highlights areas of interest within the digital pathology image. A heatmap may indicate portions of an image that depict or correlate to a particular condition or diagnosis and may indicate the accuracy or statistical confidence of such indication(s). FIG. 4A illustrates an example heatmap 400 and a detailed view 405 of the same heatmap. The heatmap is comprised of multiple cells. The cells may correspond directly to the tiles generated from the digital pathology image or may correspond to a grouping of the tiles (e.g., if a larger number of tiles are produced than would be useful for the heatmap). Each cell is assigned an intensity value, which may be normalized across all of the cells (e.g., such that the intensity values of the cells range from 0 to 1, 0 to 100, etc.). In displaying the heatmap, the intensity values of the cells may be translated to different colors, patterns, or other visual representations of intensity, etc. In the example shown in FIG. 4A, cell 407 is a high-intensity cell (represented by red tiles), and cell 409 is a low-intensity cell (represented by blue tiles). In some embodiments, color gradients may also be used to illustrate the different intensities. In particular embodiments, the intensity values of each cell may be derived from or correspond to the weighting values determined for the corresponding tile by the one or more attention networks. Thus, the heatmap may be used to quickly identify tiles of the digital pathology image that the digital pathology image processing system 210, and the weighting value generating module 213 in particular, have identified as likely including indicators of a specific condition. In the example shown in FIG. 4A, only a single heatmap has been produced. This heatmap may be based on a classification of interest, which may be one selected as the most likely condition shown in the digital pathology image or one selected by the user for review. The singular heatmap may also include a composite of weighting values generated by the one or more attention networks.

Because the weighting value generating module 213 may use multiple attention networks to generate the attention scores and weighting values, the output generating module 216 may produce an equivalent number of heatmaps (e.g., one heatmap corresponding to each classification for which the attention networks are configured to identify instances of indicators of a condition). FIG. 4B shows an example where several heatmaps 410a-410i have been produced for a single digital pathology image 415. As shown in FIG. 4B, different heatmaps displaying different colors represent the different results when the attention networks are used to identify different types of cells, cell structures, or tissue types, such as abnormal (FIG. 4B, 410a; enlarged version shown in FIG. 4C), hypertrophy (FIG. 4B, 410b; enlarged version shown in FIG. 4D), Kupffer cells (FIG. 4B, 410c; enlarged version shown in FIG. 4E), necrosis (FIG. 4B, 410d; enlarged version shown in FIG. 4F), glycogen (FIG. 4B, 410e; enlarged version shown in FIG. 4G), inflammation (FIG. 4B, 410f; enlarged version shown in FIG. 4H), lipid (FIG. 4B, 410g; enlarged version shown in FIG. 4I), peritonitis (FIG. 4B, 410h; enlarged version shown in FIG. 4J), or other (FIG. 4B, 410i; enlarged version shown in FIG. 4K). Each heatmap indicates the relative weight of tiles of the digital pathology image based on how likely the tile is to be or contain indicators of an associated condition for which the corresponding attention network.

Another example output that may be produced by the output generating module 216 is annotations for the digital pathology image. The annotations may automatically indicate areas of interest to a user (e.g., a pathologist or clinician) within the digital pathology image. As described herein, the production of annotations for digital pathology images is often a difficult and time-consuming task that requires the input of individuals with a significant amount of training. Using the weighting values generated by the attention network(s) the digital pathology image processing system 210 may identify areas that a user should focus on as contain indicators of conditions of interest. As an example, the output generating module may compare the weighting values across the set of tiles for the digital pathology image and identify the tiles that have weighting values outside the norm for the image or for images of the type. The output generating module may compare the weighting values to a threshold weighting value that may be selected by the user or may be predetermined by the digital pathology image processing system 210. The threshold may differ based on the type of condition being evaluated (e.g., the threshold value for an “abnormal” annotation may differ from a threshold value for a “necrosis” annotation). Thus, the annotations for an input digital pathology image may be based on the identification of key instances within the set of tiles for the digital pathology image.

Furthermore, the annotations may simplify the process of identifying visual matches contained within the same digital pathology image by applying pattern matching, for example drawing attention to tiles that contain the same abnormalities across the image. As embodied herein, after identifying tiles of interest (e.g., using the weighting values) the digital pathology image processing system 210 may perform gradient descent on the pixels of the identified tiles to maximize the recognition and association of tiles having similar visual characteristics as the identified tiles that may have been missed by the attention networks. Thus, the digital pathology image processing system 210 may learn and identify which visual patterns maximize the classification determination for each tile of interest. This recognition may be performed on an ad hoc basis, where new patterns are learned for each digital pathology image under consideration or may be based on a library of common patterns. For example, the digital pathology image processing system 210 may store frequently occurring patterns for each classification and proactively compare tiles to those patterns to assist with identifying tiles and areas of the digital pathology image.

Once a set of tile embeddings (e.g., 135a, 135b, . . . 135n) have been identified for annotation, the digital pathology image processing system 210 works backwards to identify the tiles corresponding to those tile embeddings. For example, each embedding may be uniquely associated with a tile, which may be identified via a tile identifier within the tile embedding. The digital pathology image processing system 210 then attempts to group proximate tiles in circumstances where a collection of tiles have been determined to showcase the same condition or indicia. Each grouping of tiles may be collected and readied for display with the relevant annotations.

A first example of a digital pathology image including annotations is shown in FIG. 5A. The digital pathology image 500 may be provided to a user device 230 (not shown) for display. The image 500 may be shown in association with the annotations 505a and 505b, which are shown as boxes drawn around the areas of interest. Thus, the viewer may easily see the context of the areas around the areas of interest. The annotations may be provided as an interactive overlay, which the user may turn on or off. Within the interface of the user device 230, the user may also perform typical functions of viewing digital pathology images, such as zooming, panning, etc.

A second example of a digital pathology image including annotations is shown in FIG. 5B. The digital pathology image 510 is shown with an interactive overlay that highlights portions of the image. The highlights, e.g., area 515a, 515b, and 515c may be shown with color coding or other visual indicia denoting similarities and differences between the highlighted areas. For example, areas 515b and 515c may be shown with the same color and be shown distinct from area 515a. This may indicate, for example, that areas 515b and 515c are associated with a first condition while area 515a is associated with a second condition. The color coding may also be used, for example, to indicate to a user that there is detailed information available for the areas or that the user has already viewed a report on the area. The overlay interface may be interactive. For example, a user may select an area, such as area 515c using an appropriate user input device of the user device 230. Upon detecting an area selection, the overlay may provide additional details about the area for review by the user. As illustrated, the user has selected area 515c. Upon detecting the user's selection, the digital pathology image processing system 210 may prompt the information box 525 to be displayed in the user interface of the user device 230. The information box may include a variety of information associated with the area 515c. For example, the information box may provide a detailed report on the detected condition and the level of confidence of the information processing system 210 in this condition. The information box may provide information about the tiles making up the area 515c, including, but not limited to, the number of tiles in the area, the approximate size of the area (absolute or relative to the sample), that other tiles showing a similar condition have been detected, and other suitable information. The information box may further provide information about the tissues depicted in the area, including by way of example only and not limitation, area size, cell size, nuclei size, distance between cells in the area, distance between nuclei in the area, distance between different cells types (e.g., distance between inflamed cells and normal cells, distance between inflamed cells and tumor cells, etc.), distance between regions exhibiting a particular condition (e.g., distance between necrotic regions within an area), and distance between one or more cells in the region to a different type of tissue or object (e.g., distance between a cell and nearest blood vessel, etc.).

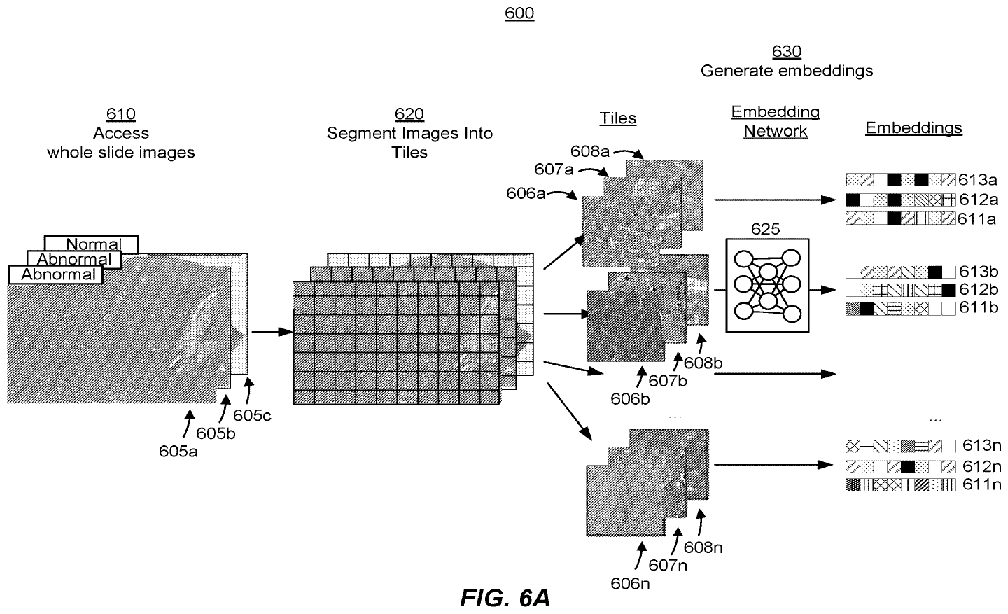

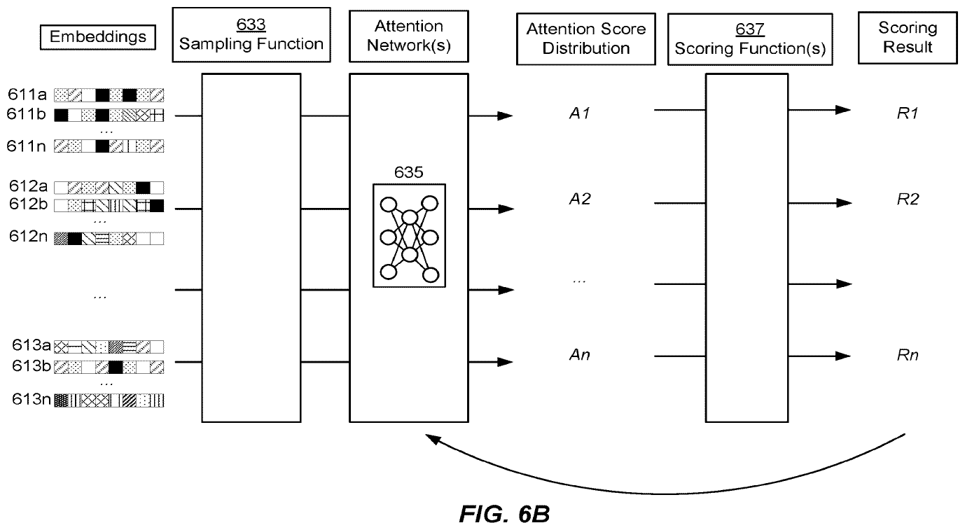

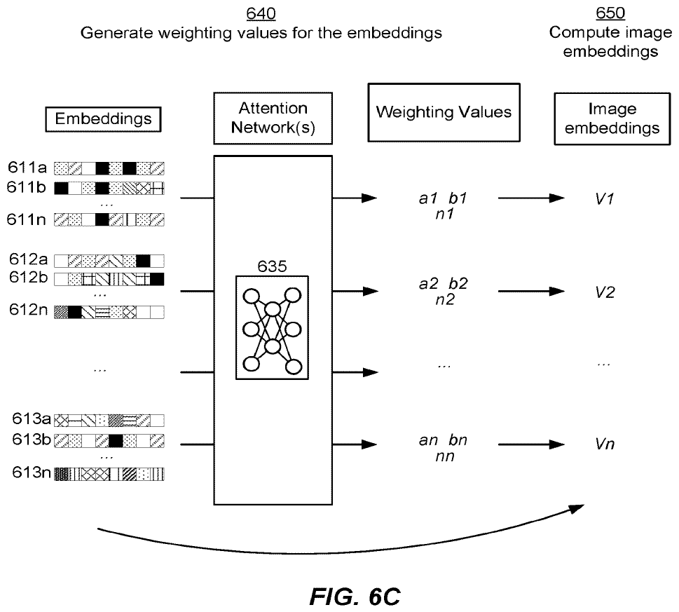

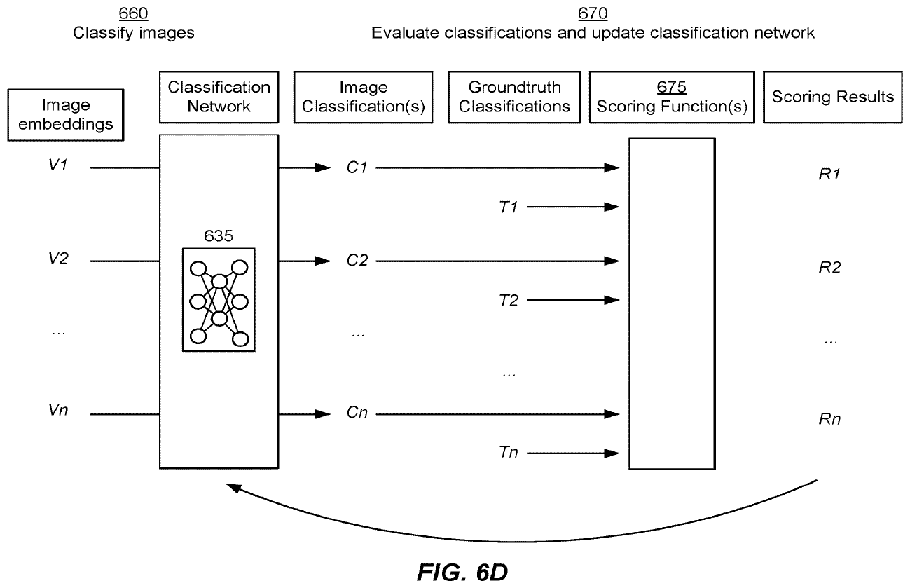

FIGS. 6A-6D illustrate an example process 600 for training the digital pathology image processing system 210 and in particular for training the attention networks used for generating weighting values and for training the classification networks that are used by the various subsystems and modules of the digital pathology image processing system 210. In general, the training process involves providing training data (e.g., whole slide images) with ground truth labels to the digital pathology image processing system 210, causing the attention networks to learn to identify key instances (e.g., tiles) that differentiate normal data from abnormal data, and causing the classification networks to learn to identify tile embedding values that positively correspond to classifications of interest. The integrated usage of the various networks and models is particularly advantageous with digital pathology images such as large whole slide images because the relatively unstructured learning approach starts with generally available labelling (e.g., normal and abnormal) and learns to identify abnormal tissue in tiles and classifications thereof. This reduces the burdens required in identifying the location of abnormal tissue, generating annotations, and making positive classifications thereof.

The model for this type of learning structure may be referred to as multiple instance learning. In multiple instance learning a collection of instances are provided together as a set with a label. Note that the individual instances are often not labelled, just the set. The label is typically based on a condition being present. The basic assumption in the multiple instance learning techniques employed by the system described is that when a set is labelled as having the condition present (e.g., when a whole slide image is labelled as abnormal) then at least one instance in the set is abnormal. Conversely, when the set is labelled as not having the instance (e.g., when a whole slide image is labelled as normal) then no instance in the set is abnormal. From this principle, and iterative training approaches, the attention network(s) may learn to identify the features of a tile (or, more specifically, a tile embedding) that correlate to an abnormal slide.

A training controller 217 of the digital pathology image processing system 210 may control training of the one or more models (e.g., neural networks) and/or functions used by digital pathology image processing system 210. In some instances, multiple or all of the neural networks used by digital pathology image processing system 210 (e.g., attention network(s) used to generate tile embeddings, a network used to generate weighting values, a network used to classify images based on image embeddings) are trained together by training controller 217. In some instances, the training controller 217 may selectively train the models using by the digital pathology image processing system 210. For example, the digital pathology image processing system 210 may use a preconfigured model to generate tile embeddings and focus on training attention network(s) to generate weighting values.

As illustrated in FIG. 6A, at 610 training controller 217 may select, retrieve, and/or access training data that includes a set of digital pathology images (e.g., whole slide images 605a, 605b, and 605c). The training data further includes a corresponding set of labels (e.g., “abnormal”, “abnormal”, “normal” respectively). At 620 the training controller 217 causes the digital pathology image processing system 210, for example using a tile-generating module 211, to segment each whole slide image into a plurality of tiles. For example, as illustrated in FIG. 6A, whole slide image 605a is segmented into tiles 606a, 606b, . . . , 606n, whole slide image 605b is segmented into tiles 607a, 607b, . . . , 607n, and whole slide image 605c is segmented into tiles 608a, 608b, . . . , 608n. For training purposes, the tiles that were segmented from whole slide images that have been labeled as abnormal are also labeled as abnormal. At 630, the training controller 217 causes the digital pathology image processing system 210, for example using a tile embedding module 212, to generate embeddings for each tile of the plurality of tiles using an embedding network 625. For example, as illustrated in FIG. 6A, the tile embedding module 212 generates, embedding 611a for tile 606a, embedding 612a for tile 607a, embedding 613a for tile 608a, embedding 611b for tile 606b, embedding 612b for tile 607b, embedding 613b for tile 608b, embedding 611n for tile 606n, embedding 612n for tile 607n, and embedding 613n for tile 608n.

FIG. 6B illustrates a process for training the attention network(s) of the weighting value generating module 213 to identify key instances (e.g., high attention value) from the embeddings generated from each whole slide image. The process will be repeated many times, which each training cycle referred to as an epoch. For simplicity, the process is illustrated using only one attention network 635, but the same techniques may be applied to multiple attention networks simultaneously. During each epoch, a randomly sampled selection of embeddings from each whole slide image are provided as input to the attention network 635. For example, the training controller 217 may use a sampling function 633 to select the set of embeddings to be used for each epoch. The attention network 635 generates attention scores A₁, A₂, . . . Aₙ for the embeddings from each sampled selection.

The training controller 217 uses one or more loss or scoring functions 637 to evaluate the attention scores generated during the epoch. Training controller 217 may use a loss function that penalizes variability or differences in attention scores across the embeddings corresponding to each individual image. Building on the intuition that the differences between “normal” tiles of “normal” and “abnormal” images should be minimized, the loss function may penalize differences between a distribution of attention scores generated for each random sampling and a reference distribution. The reference distribution may include (for example) a delta distribution (e.g., a Dirac delta function) or a uniform or Gaussian distribution. Preprocessing of the reference distribution and/or the attention score distribution may be performed, which may include (for example) shifting one or both of the two distributions to have a same center of mass or average. It will be appreciated that, alternatively, attention scores may be preprocessed prior to generating the distribution. The loss function may characterize the differences between the distributions using (for example) Kullback-Leibler (KL) divergence. If the attention score distribution included multiple disparate peaks, the divergence with a delta distribution or uniform distribution may be more dramatic, which may result in a higher penalty. While the differences in attention scores for “normal” embeddings is minimized, the loss function may reward differences in “abnormal” tiles, effectively encouraging the attention network to learn to identify abnormal tiles from among normal tiles. Another technique may use a loss function that penalizes a lack of variability across tile attention scores. For example, a loss function may scale a penalty in an inverse manner to a K-L divergence between an attention score distribution and a delta or uniform distribution. Thus, in some instances, different types (e.g., opposite types) of loss are used for tiles associated with different labels. The results R₁, R₂, . . . , Rₙ of the loss function are provided to the attention network 635, which applies or saves modifications to the attention network 635 to optimize the scores. After the attention network 635 is modified, another training epoch begins with a randomized sample of the input tiles.

The training controller 217 determines when to cease training. For example, the training controller 217 may determine to train the attention network(s) 635 for a set number of epochs. As another example, the training controller 217 may determine to train the attention network(s) 635 until the loss function indicates that the attention networks have passed a threshold value of the divergence between the distributions. As another example, the training controller 217 may periodically pause training and provide a test set of tiles where the appropriate label is known. The training controller 217 may evaluate the output of the attention network(s) 635 against the known labels on the test set to determine the accuracy of the attention network(s) 635. Once the accuracy reaches a set threshold, the training controller 217 may cease training the attention network(s) 635.

With the attention network(s) 635 sufficiently trained to identify the key instances of a provided set of tile embeddings by associating a higher attention score to the abnormal tiles, the training controller 217 may train the classifier network(s). FIGS. 6C and 6D continue from the example illustrated in FIG. 6A once the embedding network 625 has generated the embeddings. As illustrated in FIG. 6C, at 640 training controller 217 causes the digital pathology image processing system 210, for example using a weighting value generating module 213, to generate weighting values for the embeddings from each image. For example, the weighting value generating module 213 generates weighting values a₁, b₁, . . . n₁ for embeddings 611a, 611b, . . . 611n, respectively, from image 605a, generates weighting values a₂, b₂, . . . , n₂ for embeddings 612a, 612b, . . . 612n, respectively, from image 605b, and generates weighting values a₃, b₃, . . . n₃ for embeddings 613a, 613b, . . . 613n, respectively, from image 605c. To generate the weighting values, the weighting value generating module 213 may one or more attention networks 635 to generate attention scores for the embeddings as described herein. The attention scores may be further normalized before their use as weighting values. Only a single attention network 635 is illustrated in FIG. 6C for simplicity, but several attention networks (e.g., trained to identify indicators of different conditions) may also be used.

At 650, the training controller 217 causes the digital pathology image processing system 210, for example using an image embedding module 214, to compute image embeddings V₁, V₂, . . . Vₙ for each whole slide image by combining the tile embeddings in a weighted combination, using the weighting values generated for each embedding to weight the respective embedding. For example, the image embedding V₁ for the image 605a may be generated from the embeddings 611a, 611b, 611n, in combination with weighting values a1, b1, . . . , n1, the image embedding V₂ for the image 605b may be generated from the embeddings 612a, 612b, . . . , 612n, in combination with weighting values a₂, b₂, . . . , n₂, and the image embedding Vₙ for the image 605c may be generated from the embeddings 613a, 613b, . . . , 613n, in combination with weighting values aₙ, bₙ, . . . , nₙ.

As illustrated in FIG. 6D, at 660, the training controller 217 may cause the digital pathology image processing system 210, for example using an image classification module 215, to classify the images 605a, 605b, and 605c using the image embeddings V₁, V₂, . . . Vₙ. The image embeddings are provided as input to one or more classification networks 655 to generate the classifications. For simplicity, only a single classification network is illustrated, although several classification networks may be used and trained together. The classification network 635 generates image classifications based on the image embeddings, for example, classification C₁ is generated from image embedding V₁, classification C₂ is generated from image embedding V₂, and classification Cₙ is generated from image embedding Vₙ. Where the classification network 635 is to be trained to make a binary determination that an image embedding belongs to a set class or not, multiple classification networks 635 may be trained in parallel to identify that an image embedding belongs to a range of classes.

At 670, the training controller 217 accesses the ground truth classifications for each of the images being classified. As illustrated in FIG. 6D, ground truth classification T₁ corresponds to image 605a, ground truth classification T₂ corresponds to image 605b, and ground truth classification Tₙ corresponds to image 605c. The ground truth classifications are classifications that are known to be the accurate or ideal classification. For example, the ground truth classifications may be provided as part of the dataset of training images and may be generated by a pathologist or other human operator. Much like with training the attention network(s) 635, using a loss or scoring function 675 the training controller 217 compares the image classifications to the ground truth classifications and prepares results, R₁, R₂, . . . Rₙ for each image. The scoring function 675 may penalize inaccurate classifications and reward accurate classifications. Moreover, in embodiments in which the classification network 635 produces confidence intervals, the scoring function 675 may further reinforce those confidences such that, for example, strongly confident, yet inaccurate, classifications are penalized more severely than only mildly confident classifications. The results may be fed back to the classification network(s) 635, which makes or preserves alterations to optimize the scoring results. The classification network may be trained and updated using the same set of image embeddings repeatedly until a specified number of epochs has been reached or until scoring thresholds are reached. The training controller may also perform multiple iterations to train the classification network(s) 635 using a variety of training images. The classification network may also be validated using a reserved test set of images.

In some instances, training controller 217 preferentially selects, retrieves, and/or accesses training images associated with a particular label. Thus, a training data set may be biased toward digital pathology images associated with the particular label. The training data set may be defined to include more images associated with labels indicating abnormal conditions or a specified abnormal condition (e.g., inflammation and necrosis) relative to images associated with labels indicating normal conditions. This may be done to account for the expectation that more “normal” images will be readily available, but the digital pathology image processing system 210 may be targeted to identifying abnormal images.

As described herein, the traditional process for obtaining labels for digital pathology images (e.g., whole slide images) is arduous and time-consuming. The digital pathology image processing system 210 and the methods of use and training said system described herein may be used to increase the set of images available for training the various networks of the digital pathology image processing system. For example, after an initial training pass using data with known labels (including, potentially annotations), the digital pathology image processing system 210 may be used to classify images without existing labels. The generated classifications may be verified by human agents and, should correction be needed, the digital pathology image processing system 210 (e.g., the classification network(s)) may be retrained using the new data. This cycle may repeat, with the expectation that viewer interventions will be required to improve the accuracy rate on previously unseen examples. Additionally, once a specified level of accuracy has been reached, the labels generated by the digital pathology image processing system 210 may be used as a ground truth for training, e.g., the attention networks 635 used by the weighting value generating module 213.

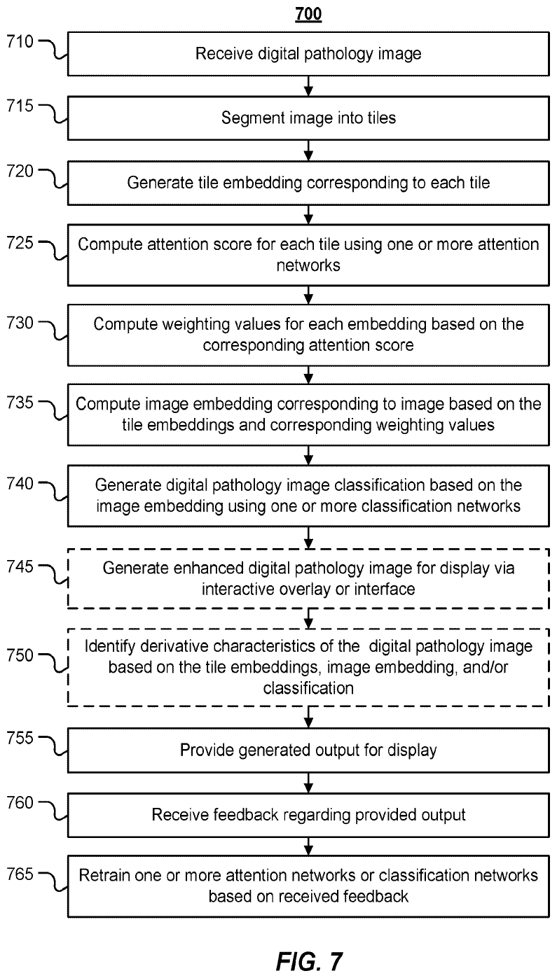

FIG. 7 illustrates an example method 700 for image classification of digital pathology images, including whole slide images, using attention networks and classification networks. The method may begin at step 710, where in digital pathology image processing system 210 receives or otherwise accesses a digital pathology image. As described herein, the digital pathology image processing system 210 may receive the image from a digital pathology image generation system directly or may receive the image from a user device 230. In other embodiments, the digital pathology image processing system 210 may be communicatively coupled with a database or other system for storing digital pathology images that facilitates the digital pathology image processing system 210 receiving the image for analysis.

At step 715, the digital pathology image processing system 210 segments the image into tiles. As described herein, the digital pathology image is expected to be significantly larger than standard images, and much larger than would normally be feasible for standard image recognition and analysis (e.g., on the order of 100,000 pixels by 100,000 pixels). To facilitate analysis, the digital pathology image processing system segments the image into tiles. The size and shape of the tile is uniform for the purposes of analysis, but the size and shape may be variable. In some embodiments, the tiles may overlap to increase the opportunity for image context to be properly analyzed by the digital pathology image processing system 210. To balance the work performed with accuracy, it may be preferable to use non-overlapping tiles. Additionally segmenting the image into tiles may involve segmenting the image based on a color channel or dominant color associated with the image.

At step 720, the digital pathology image processing system 210 generates a tile embedding corresponding to each tile. The tile embedding may map the tile to an appropriate embedding space and may be considered representative of the features shown in the tile. Within the embedding space, tiles in spatial proximity are considered similar, while distance between tiles in the embedding space is indicative of dissimilarity. The tile embedding may be generated by an embedding network that receives tiles (e.g., images) as input and produces embeddings (e.g., vector representations) as output. The embedding network may be trained on natural (e.g., non-medical images) or may be specialized on images expected to be similar to those input into the embedding network. Using natural images increases the sophistication of available training data, while using specialized images may improve the resiliency of the embedding network and allow the image embedding network to learn to discern between finer details in the input images.

At step 725, the digital pathology image processing system 210 computes an attention score for each tile using one or more attention networks. The attention score may be generated by one or more specially-trained attention networks. The attention networks receive tile embeddings and input and produce a score for each tile embedding that indicates a relative importance of the tiles. The importance of the tile, and thus the attention score, is based on identifying tile that are dissimilar from the “normal” tile. This is based on the intuition that even in digital pathology images depicting tissue having abnormalities, the overwhelming majority of tiles will depict normal-looking tissue. Therefore, the attention network may efficiently pick out tiles embeddings (and thus tiles) that are different from the rest of the tiles in each set. Multiple attention networks may be used simultaneously, with each attention network being trained to identify tiles that are abnormal in a specific manner (e.g., depicting different types of abnormalities).

At step 730, the digital pathology image processing system 210 computes weighting values for each embedding based on the corresponding attention score. The weighting values are highly correlated with the attention scores, but may result from normalizing methods, such as applying normalizing functions (e.g., the softmax function) to balance out the values of the attention scores and facilitate comparison of attention scores across different tiles, images, and attention networks.

At step 735, the digital pathology image processing system 210 computes an image embedding corresponding to the image based on the tile embeddings and corresponding weighting values. The image embedding serves as an efficient representation of the ordinarily large-format digital pathology image without losing the context of the image (e.g., based on the attention networks identifying key tiles). The image embedding may result from a weighted combination of the tile embeddings using the weighting values as weights in the combination. In embodiments, where multiple attention networks are used to attempt to identify multiple conditions, the digital pathology image processing system 210 may generate multiple image embeddings (which may each be used to classify the image) or the digital pathology image processing system 210 may create a unified image representation based on the tile embedding and multiple sets of weighting values.

At step 740, the digital pathology image processing system 210 generates a digital pathology image classification based on the image embedding using one or more classification networks. The classification networks may include artificial neural networks that receive image embeddings as input and produce either a predicted classification of the image (e.g., normal, abnormal, depicting inflammation, etc.) or a determination that the image belongs to a specified classification (e.g., in embodiments in which multiple classification networks are used and each is trained to identify a single classification for the image). The classification networks may also produce confidence scores or intervals for the detected classifications that may indicate the degree of certainty of the classification networks. The digital pathology image processing system 210 is not limited to the number or types of classifications that may be added to the digital pathology image processing system, thus as additional training samples for a new classification are identified, the capabilities of the digital pathology image processing system may be expanded in a semi-modular fashion.

At step 745, the digital pathology image processing system 210 may generate an enhanced overlay or interactive interface for the digital pathology image. The enhanced overlay or interactive interface may include visualizations of the digital pathology image designed to enhance the understanding of a viewer of the image while also providing insight to the inner-workings of the digital pathology image processing system. For example, as described herein, the digital pathology image processing system 210 may produce one or more “heatmaps” of the digital pathology image that map to the tiles (or related groupings) of the digital pathology image. The intensity of the cells of the heatmaps may correspond to, for example, the attention scores or weighting values produced by the attention networks. Thus, multiple heatmaps may be created (one corresponding to each of the attention networks) or a single heatmap may be created that illustrates the output of the various attention networks. The digital pathology image processing system 210 may also produce annotations for the digital pathology image that identify areas of the image that may be interesting to the viewer. For example, using the attention scores or weighting values, the digital pathology image processing system 210 may identify regions of the image, indicate the classification determined by the classification network, of the tiles associated with that region, and provide additional data regarding that region and the tiles within. The system may also use the tiles within an annotation feature to perform image analysis and recognition on other tiles in the image, indicating where similar features may be found. These forms of output, and many others, may be designed to be provided through the user device 230.

At step 750, the digital pathology image processing system 210 may identify derivative characteristics of the digital pathology image or the tissues depicted therein based on the tile embeddings, image embeddings, and/or classification. For example, the digital pathology image processing system 210 may store associations and correlations between certain types of classifications or features captured in tile embeddings. For example, the digital pathology image processing system may learn natural associations between types of abnormalities that may be depicted in digital pathology images. Thus, the derivative characteristics may serve as warning or reminders to the user to look for additional features in the digital pathology image. The derivative characteristics may also correlate tile embeddings across digital pathology images. For example, the digital pathology image processing system 210 may store tile embeddings or patterns of tile embeddings and perform pattern matching with an image being evaluated to draw attention to the similarities between previously-reviewed images. The digital pathology image processing system 210 may therefore serve as a tool to identify underlying similarities and characteristics.

At step 755, the digital pathology image processing system 210 provides the generated output for display. The generated output may include, for example, the digital pathology image classification, the enhance overlay or interactive interface, or the derivative characteristics and statistics thereon. These output and more may be provided to a user via, for example, a suitably configured user device 230. The output may be provided in an interactive interface that facilitates the user reviewing the analysis performed by the digital pathology image processing system 210 while also supporting the user's independent analysis. For example, the user may turn various features of the output on or off, zoom, pan, and otherwise manipulate the digital pathology image, and provide feedback or notes regarding the classifications, annotations, and derivative characteristics.

At step 760, the digital pathology image processing system 210 may receive feedback regarding the provided output. The user may provide feedback regarding the accuracy of the classifications or annotations. The user can, for example, indicate areas of interest to the user (as well as the reason why they are interesting) that were not previously identified by the digital pathology image processing system 210. The user may additionally indicate additional classifications for the image that were not already suggested or captured by the digital pathology image processing system 210. This feedback may also be stored for the user's later access, for example as clinical notes.

At step 765, the digital pathology image processing system 210 use the feedback to retrain one or more of the networks, for example, the attention networks or classification networks, used in generated the classification. The digital pathology image processing system 210 may use the feedback to supplement the dataset available to the digital pathology image processing system 210 with the additional benefit that the feedback has been provided by a human expert which increases its reliability. The digital pathology image processing system 210 may continuously revise the networks underlying the analysis provided by the system with a goal of increasing the accuracy of its classifications as well as increasing the rate at which the digital pathology image processing system identifies major areas of interest (e.g., attributes high attention scores to highly descriptive tiles). Thus, the digital pathology image processing system 210 is not a static system, but may offer and benefit from continuous improvement.

Particular embodiments may repeat one or more steps of the method of FIG. 7, where appropriate. Although this disclosure describes and illustrates particular steps of the method of FIG. 7 as occurring in a particular order, this disclosure contemplates any suitable steps of the method of FIG. 7 occurring in any suitable order. Moreover, although this disclosure describes and illustrates an example method for image classification of digital pathology images using attention networks and classification networks including the particular steps of the method of FIG. 7, this disclosure contemplates any suitable method for image classification of digital pathology images using attention networks and classification networks including any suitable steps, which may include all, some, or none of the steps of the method of FIG. 7, where appropriate. Furthermore, although this disclosure describes and illustrates particular components, devices, or systems carrying out particular steps of the method of FIG. 7, this disclosure contemplates any suitable combination of any suitable components, devices, or systems carrying out any suitable steps of the method of FIG. 7.

The general techniques described herein may be integrated into a variety of tools and use cases. For example, as described, a user (e.g., pathology or clinician) may access a user device 230 that is in communication with the digital pathology image processing system 210 and provide a digital pathology image for analysis. The digital pathology image processing system 210, or the connection to the digital pathology image processing system may be provided as a standalone software tool or package that automatically annotates digital pathology images and/or generates heatmaps evaluating the images under analysis. As a standalone tool or plug-in that may be purchased or licensed on a streamlined basis, the tool may be used to augment the capabilities of a research or clinical lab. Additionally, the tool may be integrated into the services made available to the customer of digital pathology image generation systems. For example, the tool may be provided as a unified workflow, where a user who conducts or requests a digital pathology image to be created automatically receives an annotated image or heatmap equivalent. Therefore, in addition to improving digital pathology image analysis, the techniques may be integrated into existing systems to provide additional features not previously considered or possible.

Moreover, the digital pathology image processing system 210 may be trained and customized for use in particular settings. For example, the digital pathology image processing system 210 may be specifically trained for use in providing clinical diagnoses relating to specific types of tissue (e.g., lung, heart, blood, liver, etc.). As another example, the digital pathology image processing system 210 may be trained to assist with safety assessment, for example in determining levels or degrees of toxicity associated with drugs or other potential therapeutic treatments. Once trained for use in a specific subject matter or use case, the digital pathology image processing system 210 is not necessarily limited to that use case. For example, the digital pathology image processing system may be trained for use in toxicity assessment for liver tissues, but the resulting models may be applied to a diagnostic setting. Training may be performed in a particular context, e.g., toxicity assessment, due to a relatively larger set of at least partially labeled or annotated digital pathology images. The included appendix relates to results of using the techniques described herein to perform toxicity assessment, including identifying a common toxicity event, and illustrate example output related to toxicity assessment.

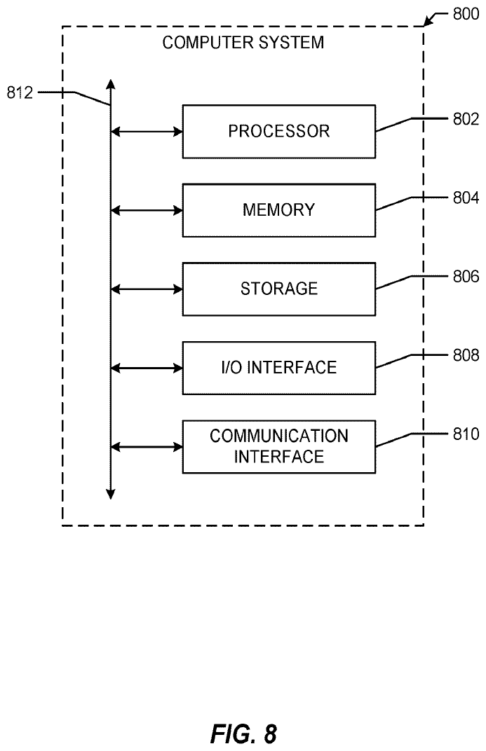

FIG. 8 illustrates an example computer system 800. In particular embodiments, one or more computer systems 800 perform one or more steps of one or more methods described or illustrated herein. In particular embodiments, one or more computer systems 800 provide functionality described or illustrated herein. In particular embodiments, software running on one or more computer systems 800 performs one or more steps of one or more methods described or illustrated herein or provides functionality described or illustrated herein. Particular embodiments include one or more portions of one or more computer systems 800. Herein, reference to a computer system may encompass a computing device, and vice versa, where appropriate. Moreover, reference to a computer system may encompass one or more computer systems, where appropriate.

This disclosure contemplates any suitable number of computer systems 800. This disclosure contemplates computer system 800 taking any suitable physical form. As example and not by way of limitation, computer system 800 may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, an augmented/virtual reality device, or a combination of two or more of these. Where appropriate, computer system 800 may include one or more computer systems 800; be unitary or distributed; span multiple locations; span multiple machines; span multiple data centers; or reside in a cloud, which may include one or more cloud components in one or more networks. Where appropriate, one or more computer systems 800 may perform without substantial spatial or temporal limitation one or more steps of one or more methods described or illustrated herein. As an example and not by way of limitation, one or more computer systems 800 may perform in real time or in batch mode one or more steps of one or more methods described or illustrated herein. One or more computer systems 800 may perform at different times or at different locations one or more steps of one or more methods described or illustrated herein, where appropriate.

In particular embodiments, computer system 800 includes a processor 802, memory 804, storage 806, an input/output (I/O) interface 808, a communication interface 810, and a bus 812. Although this disclosure describes and illustrates a particular computer system having a particular number of particular components in a particular arrangement, this disclosure contemplates any suitable computer system having any suitable number of any suitable components in any suitable arrangement.

In particular embodiments, processor 802 includes hardware for executing instructions, such as those making up a computer program. As an example and not by way of limitation, to execute instructions, processor 802 may retrieve (or fetch) the instructions from an internal register, an internal cache, memory 804, or storage 806; decode and execute them; and then write one or more results to an internal register, an internal cache, memory 804, or storage 806. In particular embodiments, processor 802 may include one or more internal caches for data, instructions, or addresses. This disclosure contemplates processor 802 including any suitable number of any suitable internal caches, where appropriate. As an example and not by way of limitation, processor 802 may include one or more instruction caches, one or more data caches, and one or more translation lookaside buffers (TLBs). Instructions in the instruction caches may be copies of instructions in memory 804 or storage 806, and the instruction caches may speed up retrieval of those instructions by processor 802. Data in the data caches may be copies of data in memory 804 or storage 806 for instructions executing at processor 802 to operate on; the results of previous instructions executed at processor 802 for access by subsequent instructions executing at processor 802 or for writing to memory 804 or storage 806; or other suitable data. The data caches may speed up read or write operations by processor 802. The TLBs may speed up virtual-address translation for processor 802. In particular embodiments, processor 802 may include one or more internal registers for data, instructions, or addresses. This disclosure contemplates processor 802 including any suitable number of any suitable internal registers, where appropriate. Where appropriate, processor 802 may include one or more arithmetic logic units (ALUs); be a multi-core processor; or include one or more processors 802. Although this disclosure describes and illustrates a particular processor, this disclosure contemplates any suitable processor.

In particular embodiments, memory 804 includes main memory for storing instructions for processor 802 to execute or data for processor 802 to operate on. As an example and not by way of limitation, computer system 800 may load instructions from storage 806 or another source (such as, for example, another computer system 800) to memory 804. Processor 802 may then load the instructions from memory 804 to an internal register or internal cache. To execute the instructions, processor 802 may retrieve the instructions from the internal register or internal cache and decode them. During or after execution of the instructions, processor 802 may write one or more results (which may be intermediate or final results) to the internal register or internal cache. Processor 802 may then write one or more of those results to memory 804. In particular embodiments, processor 802 executes only instructions in one or more internal registers or internal caches or in memory 804 (as opposed to storage 806 or elsewhere) and operates only on data in one or more internal registers or internal caches or in memory 804 (as opposed to storage 806 or elsewhere). One or more memory buses (which may each include an address bus and a data bus) may couple processor 802 to memory 804. Bus 812 may include one or more memory buses, as described below. In particular embodiments, one or more memory management units (MMUs) reside between processor 802 and memory 804 and facilitate accesses to memory 804 requested by processor 802. In particular embodiments, memory 804 includes random access memory (RAM). This RAM may be volatile memory, where appropriate. Where appropriate, this RAM may be dynamic RAM (DRAM) or static RAM (SRAM). Moreover, where appropriate, this RAM may be single-ported or multi-ported RAM. This disclosure contemplates any suitable RAM. Memory 804 may include one or more memories 804, where appropriate. Although this disclosure describes and illustrates particular memory, this disclosure contemplates any suitable memory.

In particular embodiments, storage 806 includes mass storage for data or instructions. As an example and not by way of limitation, storage 806 may include a hard disk drive (HDD), a floppy disk drive, flash memory, an optical disc, a magneto-optical disc, magnetic tape, or a Universal Serial Bus (USB) drive or a combination of two or more of these. Storage 806 may include removable or non-removable (or fixed) media, where appropriate. Storage 806 may be internal or external to computer system 800, where appropriate. In particular embodiments, storage 806 is non-volatile, solid-state memory. In particular embodiments, storage 806 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 806 taking any suitable physical form. Storage 806 may include one or more storage control units facilitating communication between processor 802 and storage 806, where appropriate. Where appropriate, storage 806 may include one or more storages 806. Although this disclosure describes and illustrates particular storage, this disclosure contemplates any suitable storage.

In particular embodiments, I/O interface 808 includes hardware, software, or both, providing one or more interfaces for communication between computer system 800 and one or more I/O devices. Computer system 800 may include one or more of these I/O devices, where appropriate. One or more of these I/O devices may enable communication between a person and computer system 800. As an example and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 808 for them. Where appropriate, I/O interface 808 may include one or more device or software drivers enabling processor 802 to drive one or more of these I/O devices. I/O interface 808 may include one or more I/O interfaces 808, where appropriate. Although this disclosure describes and illustrates a particular I/O interface, this disclosure contemplates any suitable I/O interface.

In particular embodiments, communication interface 810 includes hardware, software, or both providing one or more interfaces for communication (such as, for example, packet-based communication) between computer system 800 and one or more other computer systems 800 or one or more networks. As an example and not by way of limitation, communication interface 810 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 810 for it. As an example and not by way of limitation, computer system 800 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 800 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. Computer system 800 may include any suitable communication interface 810 for any of these networks, where appropriate. Communication interface 810 may include one or more communication interfaces 810, where appropriate. Although this disclosure describes and illustrates a particular communication interface, this disclosure contemplates any suitable communication interface.

In particular embodiments, bus 812 includes hardware, software, or both coupling components of computer system 800 to each other. As an example and not by way of limitation, bus 812 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 812 may include one or more buses 812, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend. Furthermore, reference in the appended claims to an apparatus or system or a component of an apparatus or system being adapted to, arranged to, capable of, configured to, enabled to, operable to, or operative to perform a particular function encompasses that apparatus, system, component, whether or not it or that particular function is activated, turned on, or unlocked, as long as that apparatus, system, or component is so adapted, arranged, capable, configured, enabled, operable, or operative. Additionally, although this disclosure describes or illustrates particular embodiments as providing particular advantages, particular embodiments may provide none, some, or all of these advantages.

What is claimed is:

1. A computer-implemented method comprising:
receiving a whole slide image;
segmenting the whole slide image into a plurality of tiles;
generating a feature vector for each of the tiles, wherein the feature vector for each of the tiles represents an embedding for the tile;
computing a weighting value corresponding to each of the feature vectors using an attention network;
computing an image embedding based on the feature vectors, wherein each of the feature vectors is weighted based on the weighting value corresponding to the feature vector; and
generating a classification for the whole slide image based on the image embedding.

2. The method of claim 1, further comprising:
generating a heatmap corresponding to the whole slide image, wherein the heatmap comprises a plurality of regions associated with a plurality of intensity values, respectively, wherein one or more regions of the plurality of regions is associated with an indication of a condition in the whole slide image, and wherein the respective intensity value associated with the one or more regions correlates to a statistical confidence of the indication.

3. The method of claim 1, wherein the classification for the whole slide image indicates the presence of one or more biological abnormalities in tissue depicted in the whole slide image, the one or more biological abnormalities comprising hypertrophy, Kupffer cell abnormalities, necrosis, inflammation, glycogen abnormalities, lipid abnormalities, peritonitis, anisokaryosis, cellular infiltration, karyomegaly, microgranuloma, hyperplasia, or vacuolation.

4. The method of claim 1, wherein the classification for the whole slide image includes an evaluation of a toxic event associated with tissue depicted in the whole slide image.

5. The method of claim 1, further comprising:
generating a respective classification for the whole slide image based on each attention network of a plurality of attention networks.

6. The method of claim 1, further comprising generating annotations for the whole slide image based on the weighting values by:
identifying one or more weighting values satisfying a predetermined criteria;
identifying one or more feature vectors corresponding to the identified weighting values; and
identifying one or more tiles corresponding to the identified feature vectors.

7. The method of claim 6, further comprising providing the annotations for the whole slide image for display in association with the whole slide image, wherein providing the annotations comprises marking the one or more identified tiles.

8. The method of claim 1, further comprising:
providing the classification for the whole slide image to a pathologist for verification.

9. The method of claim 1, further comprising:
calculating a confidence score associated with the classification for the whole slide image based on at least the weighting values; and
providing the confidence score for display in association with the classification for the whole slide image.

10. The method of claim 1, further comprising:
identifying, based on the feature vectors, weighting values, and slide embedding feature value, one or more derivative characteristics associated with the classification for the whole slide image.

11. The method of claim 1, further comprising:
generating a plurality of classifications for a plurality of whole slide images, respectively; and
training one or more attention networks to predict weighting values associated with one or more conditions, respectively, using the plurality of classifications.

12. The method of claim 1, wherein the classification indicates the whole slide image depicts one or more abnormalities associated with the tissue depicted in the whole slide image.

13. The method of claim 1, wherein the whole slide image is received from a user device and the method includes providing the classification for the whole slide image to the user device for display.

14. The method of claim 1, wherein the whole slide image is received from a digital pathology image generation system communicatively coupled with a digital pathology image processing system that performs the method.

15. A digital pathology image processing system comprising:
one or more processors; and
one or more computer-readable non-transitory storage media coupled to one or more of the processors and comprising instructions operable when executed by one or more of the processors to cause the system to perform operations comprising:
receiving a whole slide image;
segmenting the whole slide image into a plurality of tiles;
generating a feature vector for each of the tiles, wherein the feature vector for each of the tiles represents an embedding for the tile;
computing a weighting value corresponding to each of the feature vectors using an attention network;
computing an image embedding based on the feature vectors, wherein each of the feature vectors is weighted based on the weighting value corresponding to the feature vector; and
generating a classification for the whole slide image based on the image embedding.

16. The digital pathology image processing system of claim 15, wherein the instructions are further operable when executed by one or more of the processors to cause the system to perform operations further comprising:
generating a heatmap corresponding to the whole slide image, wherein the heatmap comprises a plurality of regions associated with a plurality of intensity values, respectively, wherein one or more regions of the plurality of regions is associated with an indication of a condition in the whole slide image, and wherein the respective intensity value associated with the one or more regions correlates to a statistical confidence of the indication.

17. The digital pathology image processing system of claim 15, wherein the classification for the whole slide image indicates the presence of one or more biological abnormalities in tissue depicted in the whole slide image, the one or more biological abnormalities comprising hypertrophy, Kupffer cell abnormalities, necrosis, inflammation, glycogen abnormalities, lipid abnormalities, peritonitis, anisokaryosis, cellular infiltration, karyomegaly, microgranuloma, hyperplasia, or vacuolation.

18. One or more computer-readable non-transitory storage media including instructions that, when executed by one or more processors, are configured to cause the one or more processors of a digital pathology image processing system to perform operations comprising:
receiving a whole slide image;
segmenting the whole slide image into a plurality of tiles;
generating a feature vector for each of the tiles, wherein the feature vector for each of the tiles represents an embedding for the tile;
computing a weighting value corresponding to each of the feature vectors using an attention network;
computing an image embedding based on the feature vectors, wherein each of the feature vectors is weighted based on the weighting value corresponding to the feature vector; and
generating a classification for the whole slide image based on the image embedding.

19. The one or more computer-readable non-transitory storage media of claim 18, wherein the instructions are further configured to cause the one or more processors of the digital pathology image processing system to perform operations further comprising:
generating a heatmap corresponding to the whole slide image, wherein the heatmap comprises a plurality of regions associated with a plurality of intensity values, respectively, wherein one or more regions of the plurality of regions is associated with an indication of a condition in the whole slide image, and wherein the respective intensity value associated with the one or more regions correlates to a statistical confidence of the indication.

20. The one or more computer-readable non-transitory storage media of claim 18, wherein the classification for the whole slide image indicates the presence of one or more biological abnormalities in tissue depicted in the whole slide image, the one or more biological abnormalities comprising hypertrophy, Kupffer cell abnormalities, necrosis, inflammation, glycogen abnormalities, lipid abnormalities, peritonitis, anisokaryosis, cellular infiltration, karyomegaly, microgranuloma, hyperplasia, or vacuolation.
